SEQUENCE LISTING (23 sequences)

SEQ ID NO: 1 - DNA, 171 bp, Nicotiana tabacum
atggcttcctcagttctttcctctgcagcagttgccacccgcagcaatgttgctcaagctaacatggttgcacctttcactggccttaagtcagctgcctcattccctgtttcaaggaagcaaaaccttgacatcacttccattgccagcaacggcggaagagtgcaatgc

SEQ ID NO: 2 - protein, 57 aa, Nicotiana tabacum
MASSVLSSAAVATRSNVAQANMVAPFTGLKSAASFPVSRKQNLDITSIASNGGRVQC

SEQ ID NO: 3 - DNA, 1450 bp, Artificial
  L1 gene from canine oral papillomavirus (COPV) with codon optimized for expression in tobacco plants
atggctgtttggcttcctgctcagaataagttctatcttccacctcaaccatctactaaggttctttctactgatgaatatgtttctagaactaatatcttctatcatgcttcatctgaaagattgcttactgttggtcatccattctatgaaatttataaggaagagagatctgaagaggttattgttccaaaggtttctccaaatcaatatagagtgtttagattgcttcttccagatcctaataactttgctttcggtgataagtcattgtttgatccagagaaggaaaggcttgtttggggtcttagaggtcttgaaattggaagaggtcaaccacttggtatttctgttaccggtcatccaacatttgatagatataatgatgttgagaatccaaacaagaatcttgctggtcatggaggtggaactgattctagagttaatatgggtcttgatccaaagcaaactcaaatgtttatgattggatgtaagccagctcttggtgaacattggtctcttactagatggtgtactggtcaagttcatactgcaggacaatgcccacctattgaacttagaaatactacaattgaagatggtgatatggttgatattggatttggtgctatggatttcaaggctcttcaacattataagtctggtgttccaattgatattgttaattctgcttgtaagtatccagattatcttaagatggctaatgaaccttatggtgatagatgcttcttctttgttaggagagagcaactttacgctaggcacattatgtctagatctggtactcaaggtcttgaacctgttccaaaggatacttatgctactagagaagacaacaatattggtacaactaattacttctctactccatcaggttctcttgtttcatctgaaggtcaattgtttaatagaccatattggattcaaagatctcaaggtaagaacaatggtattgcttggggtaatcaattgtttcttactgttgtggataatactagaggtacaccacttactattaatattggtcaacaggataagccagaggaaggtaattatgtgccatcttcatatagaacttatcttagacatgttgaagagtatgaagtttctattattgttcaactttgtaaggttaagctttctccagagaatcttgctatcattcatactatggatccaaatatcattgaagattggcatcttaatgttactcctccatctggtactcttgatgatacttatagatatattaattctcttgctactaagtgtccaactaatattcctccaaagactaatgttgatccatttgctgatttcaagttctgggaagttgatcttaaggataagatgactgaacaacttgatcaaactccacttggtagaaagttcttgtttcaaactaacgttcttagaccaaggtctgtta

SEQ ID NO: 4 - DNA, 130 bp, Artificial
  a modified tobacco etch virus (TEV) nucleotide sequence for ER/apoplast targeting
ctcaacacaacatatacaaaacaaacgaatctcaagcaatcaagcattctacttctattgcagcaatttaaatcatttcttttaaagcaaaagcaattttctgaaaattttcaccatttacgaacgatag

SEQ ID NO: 5 - protein, 43 aa, Artificial
  a modified TEV peptide sequence for ER/apoplast targeting
LNTTYTKQTNLKQSSILLLLQQFKSFLLKQKQFSENFHHLRTI

SEQ ID NO: 6 - DNA, 90 bp, Artificial
  a modified tobacco nucleotide sequence for ER/apoplast targeting
atgggatttgttctcttttcacaattgccttcatttcttcttgtctctacacttctcttattcctagtaatatcccactcttgccgtgcc

SEQ ID NO: 7 - protein, 30 aa, Artificial
  a modified tobacco peptide sequence for ER/apoplast targeting
MGFVLFSQLPSFLLVSTLLLFLVISHSCRA

SEQ ID NO: 8 - DNA, 30 bp, Artificial
  primer
ctcgagatggcttcctcagttctttcctct

SEQ ID NO: 9 - DNA, 26 bp, Artificial
  primer
gcattgcactcttccgccgttgctgg

SEQ ID NO: 10 - DNA, 31 bp, Artificial
  primer
ctcgagatggcttcctcagttctttcctctg

SEQ ID NO: 11 - DNA, 40 bp, Artificial
  primer
agcaggaagccaaacagccatgcattgcactcttccgccg

SEQ ID NO: 12 - DNA, 31 bp, Artificial
  primer
accggtaacagaaataccaagtggttgacct

SEQ ID NO: 13 - DNA, 171 bp, Pisum sativum
atggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgc

SEQ ID NO: 14 - DNA, 171 bp, Lactuca sativa
atggcctccatctcctcctcagccatcgccaccgtcaaccggaccacctccacccaagctagcttggcagctccattcaccggcctcaagtctaacgtagctttcccagttaccaagaaggctaacaatgacttttcatccctacccagcaacggtggaagagtacaatgc

SEQ ID NO: 15 - DNA, 135 bp, Oryza sativa
atggccccctccgtgatggcgtcgtcggccaccaccgtcgctcccttccagggctcaagtccaccgccggcatgccgtcgcccgccgtccgaactccagcttcggcaacgtcagcatggcggcaggatcaggtgc

SEQ ID NO: 16 - protein, 58 aa, Pisum sativum
MASMISSSAVTTVSRASTVQSAAVAPFGGLKSMTGFPVKKVNTDITSITSNGGRVKCM

SEQ ID NO: 17 - protein, 58 aa, Lactuca sativa
MASISSSAIATVNRTTSTQASLAAPFTGLKSNVAFPVTKKANNDFSSLPSNGGRVQCM

SEQ ID NO: 18 - protein, 58 aa, Nicotiana tabacum
MASSVLSSAAVATRSNVAQANMVAPFTGLKSAASFPVSRKQNLDITSIASNGGRVQCM

SEQ ID NO: 19 - protein, 56 aa, Arabidopsis thaliana
MASSMLSSTAVVTSPAQATMVAPFTGLKSSASFPVTRKANNDITSITSNGGRVSCM

SEQ ID NO: 20 - protein, 58 aa, Spinacia oleracea
MASSVLSSAAVATVSRTPAQASMVAPFTGLKSTVGFPATKKNDDITSLASNGGRVQCM

SEQ ID NO: 21 - protein, 47 aa, Triticum aestivum
MAPAVMASSATTVAPFQGLKSTAGLPVSRRSRGSLGSVSNGGRIRCM

SEQ ID NO: 22 - protein, 48 aa, Oryza sativa
MAPTVMASSATSVAPFQGLKSTAGLPVSRRSTNSGFGNVSNGGRIKCM

SEQ ID NO: 23 - DNA, 1512 bp, Canine oral papillomavirus
atggcggtttggcttcctgcacagaataaattttaccttccaccacagcccagcaccaaggtcttaagcacggatgaatatgtctccagaacaaatattttttatcatgctagcagtgaacgtcttcttactgtggggcaccctttttatgaaatttataaagaagaacgttctgaagaggttatagttcctaaagtatctcctaatcagtaccgggtattccgcttgctacttccagaccctaacaattttgcatttggagataagtcattatttgatcctgaaaaagaaagacttgtttggggcttaagaggattagaaataggtagggggcaaccattaggtataagtgttacgggtcatccaacatttgacagatacaatgatgtagaaaacccaaacaaaaatcttgctggacatggaggtggaacagacagcagggttaacatgggtttagaccctaaacaaactcagatgtttatgatagggtgcaaaccagctttaggtgaacactggtctttaactagatggtgcacaggacaggtacacactgcaggacaatgtccaccaatagaactgagaaacacaacaatagaagatggagatatggtagatatagggtttggtgcaatggattttaaggctttgcagcattataagtcaggagttccaattgacatagtaaattctgcatgcaaatatccagactacctcaaaatggcaaatgagccttatggagatagatgttttttttttgtaagaagagagcaactgtatgccagacatattatgtccagatctggcacacaaggtttagaaccagtccccaaagatacctatgcaacaagagaagacaataacataggaacaactaattacttctccacacctagtggctctctggtttctagtgagggacaactgtttaacaggccttactggatccagcgctcgcagggcaagaataatgggattgcatggggcaatcagctgtttttaacagtagtggacaacacacgaggaactcccttaactataaacatagggcaacaagacaagccagaagaaggaaattatgttccttcatcatacagaacctacctcagacatgttgaagaatatgaagtaagcataattgtgcagctgtgcaaagttaagctgtcccctgaaaatctagcaataattcatactatggatcctaatattattgaggattggcacctaaatgtcactcctccatctggtactttagatgacacatataggtacataaactctcttgctactaagtgccctactaatatacctccaaaaactaacgttgatccttttgcagactttaaattttgggaagtagatcttaaagataaaatgactgaacagttagaccaaactccactgggtcgcaaatttttattccagacaaatgtgttacgtcctagatctgtaaaagtacgttctacctcgcacgtttctgtcaaacgaaaagctgtgaaacgcaaacgcaaataa

(19) United States
(12) Patent Application Publication
(10) Pub. No.: US 2011/0138495 A1
(43) Pub. Date: Jun. 9, 2011

(54) Methods and Compositions for Expressing Proteins In Plants

(76) Inventors: Arthur Weissinger, Raleigh, NC (US); Kasi Azhakanandam, Raleigh, NC (US); Rongda Qu, Cary, NC (US); Jennifer Nichloson Levin, Raleigh, NC (US); Sandra M. Weissinger, Raleigh, NC (US)

(21) Appl. No.: 11/988,316

(22) PCT Filed: Jul. 3, 2006
(86) PCT No.: PCT/US06/26061

§ 371 Date: Jan. 14, 2011

Related U.S. Application Data
(60) Provisional application No. 60/696,773, filed on Jul. 5, 2005.

(51) Int. Cl. C12N 15/82; C07K 14/025; A01H 5/00; C12N 5/10; C07H 21/04

(52) U.S. Cl. 800/278; 530/350; 800/298; 435/419; 536/23.4

(57) ABSTRACT

Methods and compositions comprising an expression construct and a suppressor of posttranscriptional gene silencing construct are described. The expression construct and suppressor construct may comprise a viral amplicon. The expression construct may comprise fusing the target gene to the 3′ and/or 5′ end of a gene encoding a transit peptide sequence or a signaling peptide sequences. The transit or signal peptide sequence directs the target gene product to a subcellular location. Methods comprise the production of several heterologous proteins in a single plant. The invention comprises methods for plant production and protein harvest that will yield useful amounts of the desired protein(s) in as little as one to two weeks after the initiation of the production cycle. Methods for the inoculation of recipient plants by spraying with recombinant Agrobacterium suspensions containing the constructs of interest are taught.

### RELATED APPLICATIONS

This application claims the benefit of U.S. Provisional Patent Application No. 60/696,773, filed Jul. 5, 2005, which is herein incorporated by reference in its entirety.

### GOVERNMENT INTEREST

This invention was made with U.S. Government support under contract No. 2001-38712-10592, awarded by the U.S. Department of Agriculture—Cooperative State Research, Education, and Extension Service. The Government has certain rights in this Invention.

### TECHNICAL FIELD

The invention relates to the field of producing gene products in plants. The invention relates particularly to methods and compositions for enhancing the expression of foreign or endogenous genes introduced into plants.

### BACKGROUND OF THE INVENTION

The availability of expression systems for the production of valuable recombinant proteins is an important issue in biotechnology. Some well-established expression systems to produce recombinant proteins utilize Escherichia coil or yeast fermentation systems. Other well-established expression systems involve mammalian cell systems, such as Chinese hamster ovary cells or HEK293 cells, to produce recombinant proteins. Although these systems are well-characterized and frequently utilized by researchers and industry, many of these systems have specific shortcomings that limit their use in a variety of applications. For example, bacteria cannot perform the complex post-translational modifications required for bioactivity of many commercially valuable proteins expressed from human and mammalian genes. For biological activity, many proteins of commercial and therapeutic interest require one or more post-translational modifications including proteolytic cleavage, disulfide bond formation, β-hydroxylation, γ-carboxylation, and N-linked glycosylation. Proteins expressed at high levels in bacterial expression systems frequently yield insoluble protein aggregates. On the other hand, although mammalian expression systems are capable of performing complex post-translational modifications required for the function of many human and mammalian proteins, mammalian expression systems have difficulties reaching high levels of transgene expression. Additional difficulties faced by researchers and industry using mammalian expression systems involve difficulties in scale-up and cost.

The use of plant expression systems has significant advantages compared to both bacterial, yeast and mammalian expression systems. For example, plant expression systems are easy to manipulate for introduction and expression of recombinant proteins. Plant expression systems also possess unique characteristics for safety of the isolated recombinant proteins in that plants do not serve as hosts or carriers of human pathogens. Thus, recombinant proteins that have therapeutic or medicinal value when expressed in plants are free of possible product contamination with human pathogens such as hepatitis or human immunodeficiency virus (HIV). Modern agricultural methods are capable of production of plants on a large scale. Thus, cost of production is one advantage realized with plant expression systems. Importantly, plant expression systems are capable of eukaryotic protein processing, including proteolytic cleavage, disulfide bond formation, β-hydroxylation, γ-carboxylation, and N-linked glycosylation. Tobacco (Nicotiana tabacum L.) is particularly well-suited for use as a bioreactor for production of high-value recombinant proteins. Tobacco is one of the easiest plants to transform and it is an excellent biomass producer.

Despite the distinct advantages of plant expression systems, their use for the production of commercially valuable recombinant proteins, including recombinant proteins useful for pharmaceutics, medical applications, vaccines, and industrial process, has been limited. For example, attempts to express proteins in plants have been frustrated by induction of viral-induced gene silencing or post-transcriptional gene silencing (PTGS) which has the effect of decreasing or eliminating functional expression of the desired protein. It is also known that plants express high levels of proteases in their cytoplasm or cytosolic compartments. The levels of these proteases can vary significantly depending upon growth conditions and other stimuli. The naturally occurring mixture of endogenous proteases in plants can make it difficult to express recombinant proteins. Furthermore, many desirable recombinant proteins are highly susceptible to degradation by plant proteases.

Many plant expression systems are based upon creation of stable transgenic plants. In such systems, large numbers of independently transformed plants must be generated and screened to identify those showing high-level, stable transgene expression. However, plant expression systems that are based upon the creation of stable transgenic plants have potential shortcomings limiting their use. The creation of stable transgenic plants is generally considered to be time-consuming and unpredictable. Expression levels for a desired recombinant protein can be highly variable due to genomic positional effects on the integrated transgenic construct. The use of stable transgenic expression systems makes the routine expression of complex mixtures of recombinant proteins particularly challenging. Thus, if the user requires the co-expression of two or more recombinant proteins, this can be achieved with current stable transgenic technology only by either producing complex transgenic plants, or by the time-consuming crossing of transgenic plants, each of which carries one of the transgenes of interest. This process must be followed by screening for either complex transgenics or progeny of the cross to identify plants that express all of the desired transgenic proteins. Finally, stable transgenic expression systems may not be suitable for the expression of proteins that interfere with the normal development of the plant.

What is needed are methods and compositions for expression of recombinant proteins in plants that do not require the creation of stable transgenic plants and allows for the high level expression of recombinant proteins. Ideally, plants should be made to express the desired heterologous protein by spraying the construct onto the recipient plant, or by slight wounding of the plant, followed by spraying. Methods and compositions are also required for a transient expression system that minimizes the effects of PTGS on the expression of recombinant proteins, decreases degradation of the recombinant proteins by cytosolic factors, and allows for the simultaneous production of multiple recombinant proteins in a single plant.

### SUMMARY OF THE INVENTION

The present invention comprises methods and compositions for the expression of recombinant protein products in plants. Compositions of the present invention comprise a target gene expression construct comprising a promoter operably linked to a target gene. In one aspect, a gene encoding a transit peptide is placed in-frame at the 5′ end of the gene encoding a specific desired recombinant protein. In a further aspect, the transit peptide is a peptide sequence capable of targeting the fusion protein into the chloroplasts. In another aspect, the transit peptide is removed after translocation of the fusion protein into chloroplast. In another aspect, a signal sequence is placed in-frame with the 5′ terminus of the coding sequence, and another sequence is placed in-frame with the 3′ terminus of the coding sequence, resulting in the production of a recombinant fusion protein that is retained in the endoplasmic reticulum of the plant cell. In still another aspect, a signal sequence is placed in-frame with the 5′ terminus of the coding sequence, resulting in the production of a recombinant fusion protein that is targeted to the plant apoplast.

Compositions of the present invention may comprise a PTGS suppressor construct comprising a promoter operably linked to a suppressor gene, wherein the suppressor gene is capable of suppressing post-transcriptional gene silencing (“PTGS”). In one aspect, a suppressor of PTGS is P1/HC-Pro protein encoded by a P1/HC-Pro gene. In a further aspect, a P1/HC-Pro gene is derived from tobacco etch virus. In another aspect, a P1/HC-Pro element has been altered so that plants expressing the product it encodes does not alter the plant phenotype in a deleterious fashion. In another aspect, other suppressors of PTGS derived from other viruses may be used in the compositions and methods taught herein for P1/HC-Pro or modified P1/HC-Pro elements or in methods and compositions known to those skilled in the art. In another aspect, other suppressors of PTGS derived from plants can be used in place of the P1/HC-Pro or modified P1/HC-Pro elements.

In one aspect, a target gene expression construct, a PTGS suppressor construct, or both constructs may comprise a viral amplicon.

The present invention comprises methods for the introduction into plant cells of at least one expression construct. In one aspect, at least one target gene expression construct is introduced into a transgenic plant comprising a PTGS suppressor construct. In another aspect, at least one target gene expression construct is introduced into a transgenic tobacco plant. In a further aspect, at least one target gene expression construct is introduced into a TEV-B transgenic line of Nicotiana tabacum cv. ‘Xanthi’ that expresses a form of P1/HC-Pro.

The present invention comprises methods for simultaneously introducing into plant cells at least one target gene expression construct and at least one PTGS suppressor construct. In one aspect, agroinfiltration is used to introduce at least one target gene expression construct and at least one PTGS suppressor construct into plant cells. In a further aspect, agroinfiltration is carried out using one Agrobacterium strain comprising at least one target gene expression construct mixed with a second Agrobacterium strain comprising a PTGS suppressor construct.

In one aspect, the methods of the present invention comprise harvesting plant tissue comprising the target protein, wherein the plant tissue is harvested at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 and 90 days or longer after introduction of the target gene expression construct into plant cells.

The present invention further comprises methods for the mass introduction of the expression and suppressor constructs into plants. These methods are suitable for use in applications requiring introduction of at least one target gene expression construct, at least one PTGS suppressor construct, or at least one target gene expression construct and at least one PTGS suppressor construct into plants grown in greenhouses and as crops under cultivation. Further, the methods of the present invention may be adapted to mechanized methods for use with crops under cultivation. In one aspect of the invention, Agrobacterium suspensions harboring a genetic construct of interest are delivered into a recipient plant by slightly wounding the plant followed by spraying the plant with the Agrobacterium suspension. In another aspect of the invention, Agrobacterium suspensions harboring a genetic construct of interest are delivered into a recipient plant by spraying the plant with the Agrobacterium suspension, without the need for wounding of any plant tissue.

Other features and advantages of the present invention will become apparent from the following detailed description. It should be understood, however, that the detailed description and the specific examples, while indicating the preferred embodiments of the present invention, are given by way of illustration only, since various changes and modifications within the spirit and scope of the present invention will become apparent to those skilled in the art from this detailed description.

### DESCRIPTION OF FIGURES

FIG. 1 is a schematic diagram of an exemplary amplicon construction suitable for expression of recombinant proteins in plants in accordance with one embodiment of the present invention.

FIG. 2 shows a nucleotide sequence encoding a chloroplast transit peptide (SEQ ID NO. 1).

FIG. 3 shows a peptide sequence coding for a chloroplast transit peptide (SEQ ID NO. 2).

FIG. 4 shows the nucleotide sequence for the vcp gene with codons optimized for expression in plant cells (SEQ ID NO. 3).

FIG. 5 shows a modified tobacco etch virus (TEV) nucleotide sequence for ER/apoplast targeting in tobacco plant cells (the KDEL sequence, SEQ ID NO. 4).

FIG. 6 shows a modified TEV peptide sequence for ER/apoplast targeting in tobacco plant cells (the KDEL sequence, SEQ ID NO. 5).

FIG. 7 shows a modified tobacco nucleotide sequence for ER/apoplast targeting in tobacco plant cells (the SP sequence, SEQ ID NO. 6).

FIG. 8 shows a modified tobacco peptide sequence for ER/apoplast targeting in tobacco plant cells (the SP sequence, SEQ ID NO. 7).

### DETAILED DESCRIPTION OF THE INVENTION

As used herein and in the appended claims, the singular forms “a,” “an,” and “the” include plural references, unless the content clearly dictates otherwise. Thus, for example, reference to “a plant” includes a plurality of such plants and equivalents thereof known to those skilled in the art, and reference to “the polypeptide” is a reference to one or more such polypeptides and equivalents thereof known to those skilled in the art, and so forth. All publications, patent applications, patents, and other references mentioned herein are incorporated by reference in their entirety.

The present invention comprises methods and compositions for the expression of recombinant proteins in plant cells. In particular, the compositions of the present invention comprise an expression system comprising at least one target gene expression construct and at least one PTGS suppressor construct. A target gene expression construct may comprise a promoter operably linked to a target gene constructs. In one aspect, a target gene expression construct may further comprise linking a target gene in frame with a gene encoding a peptide capable of directing the target protein to a specific subcellular compartment in plant cells. A PTGS suppressor construct may comprise a promoter operably linked to a gene encoding a suppressor of post-transcriptional gene silencing (PTGS).

The present invention is useful for and comprises compositions and methods for enhanced expression of recombinant polypeptides that may be normally unstable in plants due to sensitivity to cytosolic proteases or other factors confounding high level expression of protein in the cytosolic compartment of plant cells. By “enhanced expression” it is intended that expression of a target protein is increased over expression levels observed in conventional transgenic lines for heterologous sequences and over endogenous levels of expression for homologous sequences and as compared to expression in expression systems for recombinant proteins where expression of the recombinant target protein is subject to PTGS. It is to be understood that heterologous or exogenous sequences comprise sequences that do not occur in the plant of interest in its native state. Homologous or endogenous sequences are those that are natively present in the plant genome. Generally, expression of the target sequence is increased at least about 10%-50%, about 50%400%, about 100%-200%, and greater than 200%. Unless otherwise indicated, “polypeptide” shall include a protein, polypeptide, or peptide, and any fragment or variant or derivative thereof having polypeptide function.

The present invention comprises methods for the introduction of at least one target gene construct into plant cells. In one aspect, methods comprise introducing a target gene expression construct into plant cells simultaneously with one or more PTGS suppressor constructs. In another aspect, a target gene expression construct may be introduced into plant cells in no particular order with one or more PTGS suppressor constructs. In another aspect, a target gene expression construct may be introduced into plant cells after at least one PTGS expression construct is introduced into the plant cells. In a further aspect, a target gene expression construct is introduced into a transgenic plant, wherein the transgenic plant comprises at least one PTGS suppressor that is stably integrated into the transgenic plant genome.

The present invention comprises methods for bulk introduction of an expression system into plant cells. The methods and compositions for bulk introduction of an expression system of the present invention are suitable for both manual and mechanized application systems. In one aspect, the present invention comprises methods and compositions for bulk introduction of an expression system into stems, leaves, and whole plants, including plants grown in test plots, greenhouses, growth chambers and acreage under commercial cultivation. In another aspect, the present invention comprises methods for bulk introduction into of an expression system of the present invention wherein the target plant is slightly damaged by mechanical means, followed by spraying onto the plant a suspension of one or more Agrobacterium strains comprising one or more expression constructs and one or more suppression constructs. In one embodiment, at least one surface of leaves of a target plant are slightly damaged by at least one or more methods comprising abrasion, contusion, scratching, cutting or piercing, followed by spraying onto the plant a suspension of one or more Agrobacterium strains comprising one or more target gene expression constructs and one or more PTGS suppressor constructs. In another aspect, the present invention comprises methods for bulk introduction into of an expression system of the present invention wherein the target plant is not damaged prior to spraying onto the plant a suspension of one or more Agrobacterium strains comprising one or more expression constructs and one or more suppression constructs. In this embodiment, constructions are delivered to the recipient plant by spraying onto the plant a suspension of one or more Agrobacterium strains comprising one or more target gene expression constructs and one or more PTGS suppressor constructs.

The present invention is directed to methods for the introduction of an expression system of the present invention into plant cell culture systems, plant callus systems, embryonic plant cells, plant zygotic cells, and seeds. In one aspect, the present invention comprises methods for introduction of an expression system into plant cell culture systems.

As used herein, the term “plant” includes reference to whole plants, plant parts or organs (e.g., leaves, stems, roots, etc.), plant cells, seeds and progeny of same. “Plant cell”, as used herein, further includes, without limitation, cells obtained from or found in: seeds, suspension cultures, embryos, meristematic regions, callus tissue, leaves, roots, shoots, gametophytes, sporophytes, pollen, and microspores. Plant cells can also be understood to include modified cells, such as protoplasts, obtained from the aforementioned tissues. The class of plants which can be used in the methods of the invention is generally as broad as the class of higher plants amenable to transformation techniques, including both monocotyledonous and dicotyledonous plants.

As used herein, “chloroplast” is intended to include the various forms of plastids including, but not limited to, chloroplasts, amyloplasts, proplastids, leucoplasts, chromoplasts, and etioplasts.

As used herein, “encoding” or “encoded”, with respect to a specified nucleic acid, is intended to mean comprising the information for translation into the specified protein. A nucleic acid encoding a protein may comprise non-translated sequences (e. g., introns) within translated regions of the nucleic acid, or may lack such intervening non-translated sequences (e.g., as in cDNA). The information by which a protein is encoded is specified by the use of codons.

As used herein, “transformation” refers to the process of introducing DNA into a cell. Other terms used herein to refer to the introduction of DNA into a cell included “transformed host cell” or “transformed cell”, referring to a cell into which DNA has been introduced, and “transformed”, referring to the state of a cell after introduction of DNA into the cell. The introduced DNA is usually in the form of a vector containing an inserted piece of DNA. The introduced DNA sequence may be from the same species as the host cell or from a different species from the host cell, or it may be a hybrid DNA sequence, containing some foreign DNA and some DNA derived from the host species. In one aspect, the introduced DNA is an expression construct.

As used herein, “expression construct” is intended to mean a nucleic acid construct comprising at least one promoter operably linked to at least one target gene and any additional nucleic acid sequences as may be required for the production and use of the construct, particularly such nucleic acid sequences as may be required for the use of the nucleic acid construct into plant cells. The expression constructs of the present invention may be any of the several types known to one skilled in the art, including, but not limited to, plasmid vectors, viral-derived plasmids, including amplicons, and viral-based vectors.

As used herein, “amplicon” refers to an expression construct wherein a target gene or suppressor gene has been inserted into the cDNA of at least part of viral genome, wherein the cDNA of at least part of a viral genome has been operably linked to a promoter.

It is to be understood that an expression construct following transformation may reside in the cell in one or more subcellular locations, including, but not limited to, the cytosol, the nucleus, chloroplasts, and mitochondria. The specific subcellular location or locations of an expression construct in the cell following transformation is not believed to be critical to the function of the present invention. In one aspect, the expression construct may integrate into the host cell chromosomal DNA. In another aspect, the expression construct may remain distinct and not integrated into the host cell chromosomal DNA. In a further aspect, a fraction of the population of expression constructs introduced into the host cell may be integrated into the host cell chromosome and another fraction found in the nucleus and cytosol. However, it is not critical to know the integration status of the expression with respect to the chromosomal DNA of the host cell, nor is the integration status critical to the practice of the present invention.

As used herein, “operably linked” is intended to mean a functional linkage between two nucleic acid sequences. In one aspect, two DNA sequences such as a promoter and a second sequence may be operably linked, wherein the promoter sequence initiates and mediates RNA transcription from the second sequence. In another aspect, two DNA sequences wherein each sequence encodes a peptide, polypeptide or proteins may be operably linked, wherein the two DNA sequences are linked such that the reading frames of the two DNA sequences are in the same reading frame.

As used herein, “post-transcriptional gene silencing” or “PTGS” refers to the silencing of either exogenous or endogenous gene sequences introduced into a plant cell. It should be understood that silencing refers to a degree of reduction of production of an encoded gene product that may vary from partial to total reduction of production. Therefore, PTGS should not be taken to require complete “silencing” or elimination of production of the encoded gene product. While not wishing to be bound by any particular theory, PTGS is often understood to refer to a sequence specific RNA degradation mechanism and is a fundamental regulatory mechanism operating in diverse types of organisms.

As used herein, “PTGS suppressor construct” refers to an expression construct comprising at least one promoter operably linked to at least one gene that is a suppressor of PTGS.

As used herein, “target gene expression construct” refers to an expression construct comprising at least one promoter operably linked to at least one target gene.

As used herein, “target gene” comprises a DNA sequence encoding a specific functional product such as a protein, a RNA molecule or a nucleic acid sequence, wherein target gene comprises an exogenous or endogenous gene sequence of interest, which may be a naturally occurring gene, a synthetic gene, or a variant thereof. It is to be understood that a RNA molecule encoded by a target gene may comprise an mRNA molecule which may be translated into a target protein. It is to be further understood that a target gene may encode a RNA molecule which is not translated into a target protein, but is a RNA molecule that may comprise functions such as iRNA, suppressor RNA, tRNA, rRNA, or other functional RNA molecule.

As used herein, “target protein” comprises any peptide, polypeptide, or protein sequence that is encoded by a target gene. As used in the present invention, the term “recombinant target protein” and “target protein” may be used interchangeably and with the same meaning and effect, unless specifically specified otherwise.

Expression constructs of the present invention may comprise plant viruses to introduce and express non-viral foreign genes in plants (as taught, for example, in U.S. Pat. No. 4,855,237 and WO 9534668). In one aspect, the genome or portions of the genome of a plant virus can first be cloned into a bacterial plasmid for ease in constructing the desired viral vector with the foreign DNA. If the virus is an RNA virus, the viral RNA is generally cloned as at least one cDNA and inserted into a plasmid. The DNA plasmid may then be used to deliver the required constructions into a recipient plant or plant cell. An RNA virus may then produced by transcribing, in whole or in part as required, at least one viral sequence inserted into the plasmid and translation of one or more viral genes to produce proteins, including coat protein(s) encapsidating a viral RNA.

Expression constructs of the present invention may comprise amplicons. Suitable amplicons are taught, for example, in Sablowski et al. (1995) and Angell and Baulcombe, (1997), as well as taught in U.S. Pat. Nos. 6,635,808, 6,395,962, 5,939,541 and U.S. Patent Application Nos. 2004/0268441 and 2004/0078844. In one aspect, an amplicon comprises the cDNA of the complete genome of potato virus X (“PVX”). In another aspect, the amplicon comprises the cDNA of at least a portion of the genome of PVX. In a further aspect, the amplicon comprises the PVX genes encoding an RNA-dependent RNA polymerase, the “triple gene block genes” encoding the movement proteins 25, 12, and 8 kDa, and the coat protein, as well as a promoter operably linked to the cDNA of the PVX genome, a multiple cloning site between the “triple block genes” and the coat protein, and a transcriptional terminator following the coat protein gene. In a minimal PVX-derived amplicon, the cDNA of PVX genome comprises only the gene for RNA-dependent RNA polymerase. In another aspect, the amplicon comprises the PVX genes encoding an RNA-dependent RNA polymerase, the “triple gene block genes” encoding the movement proteins 25, 12, and 8 kDa, and a truncated gene for the coat protein. In one aspect, the promoter operably linked cDNA of the PVX genome is the 35S promoter of cauliflower mosaic virus. In another aspect, the promoter operably linked cDNA of the PVX genome is the nopaline synthase promoter of Agrobacterium tumefaciens. In another aspect, the tDNA carrying the construction(s) is operably attached at it's 3′ terminus to a nopaline synthase terminator derived from Agrobacterium tumefaciens. In one aspect, the amplicon comprises the cDNA derived from at least part of the genome tobacco rattle virus (TRV). In another aspect, the cDNA derived from at least part of the genome tobacco rattle virus (TRV) comprises the entire viral genome. In a further aspect, the cDNA derived from at least part of the genome tobacco rattle virus (TRV) comprises only the genes involved in viral replication, for example, replicase genes. In another aspect, the amplicon may comprise one or more of these elements taught herein including but not limited to, alone or in combination, the genomee of PVX, at least a portion of the genome of PVX, the PVX genes encoding an RNA-dependent RNA polymerase, the “triple gene block genes” encoding the movement proteins 25, 12, and 8 kDa, a coat protein, a promoter operably linked to cDNA of all or a portion the PVX genome, a multiple cloning site between the “triple block genes”, a coat protein, a transcriptional terminator, a truncated gene for the coat protein, a 35S promoter of cauliflower mosaic virus, a nopaline synthase promoter of Agrobacterium tumefaciens, a nopaline synthase terminator, at least part of the genome of tobacco rattle virus (TRV), the entire viral genome of tobacco rattle virus (TRV) and the the genes involved in viral replication, for example, replicase genes of tobacco rattle virus (TRV).

While not wishing to be bound by any particular theory, it is currently believed that after a cell has been transformed with DNA comprising an amplicon, a single RNA product is transcribed comprising the genes of the amplicon which then serves as an RNA template for translation. The first gene product translated is believed to be the RNA-dependent RNA polymerase, which is required for viral replication. Using the RNA product of the amplicon as a template, the RNA-dependent RNA polymerase is believed to synthesize negative strand RNA. The RNA-dependent RNA polymerase is believed to then use the negative strand RNA as a template to synthesize the various species of positive strand RNAs, which would include full-length viral genome RNA and subgenomic RNAs. The subgenomic RNAs are believed to be RNA templates for translation of internal genes within the amplicon. Among the subgenomic RNAs is the RNA transcribed from the target gene or suppressor gene. As currently understood, an amplicon permits amplification of RNAs transcribed from the genes in the amplicion. It is further understood, that if the appropriate genes are present, an amplicon may permit assembly of viral particles which can move and infect cells adjacent to the transformed cell.

The expression constructs of the present invention may be assembled from one or more other expression vectors, wherein the sequences of interest are assembled into the desired expression vector by methods known to one skilled in the art. The expression construct may further comprise 5′ and 3′ regulatory sequences operably linked to the promoter, the target gene, or to both. The expression construct may further comprise a plurality of restriction sites for insertion of the sequences of the invention to be under the transcriptional regulation of the regulatory regions. The expression construct may additionally contain prokaryotic or eukaryotic selectable marker genes.

The expression construct may comprise in the 5′-3′ direction of transcription, a transcriptional and translational initiation region, a DNA sequence of the invention, and a transcriptional and translational termination region functional in plants. The transcriptional initiation region, comprising a promoter, may be native or analogous, or foreign or heterologous, to the plant host. Additionally, the promoter may be the natural sequence or alternatively a synthetic sequence. By “foreign” is intended that the transcriptional initiation region is not found in the native plant into which the transcriptional initiation region is introduced.

The expression construct may further comprise a transcriptional termination region which may be native with the transcriptional initiation region, may be native with the operably linked DNA sequence of interest, or may be derived from another source. Convenient termination regions are available from the Ti-plasmid of Agrobacterium tumefaciens, such as the octopine synthase and nopaline synthase termination regions. See also Guerineau et al., (1991); Proudfoot (1991); Sanfacon et al., (1991); Mogen et al. (1990); Munroe et al. (1990); Ballas et al., (1989); and Joshi et al. (1987).

The expression construct may additionally contain 5′ leader sequences operably linked to the target gene. Such leader sequences can act to enhance translation. Translation leaders are known in the art and include: picornavirus leaders, for example, the EMCV leader (Encephalomyocarditis 5′ noncoding region), Elroy-Stein et al. (1989; potyvirus leaders, for example, TEV leader (Tobacco Etch Virus), Allison et al. (1986); human immunoglobulin heavy-chain binding protein (BiP), Macejak et al. (1991); untranslated leader from the coat protein mRNA of alfalfa mosaic virus (AMV RNA 4), Jobling et al., (1987); tobacco mosaic virus leader (TMV), Gallie et al. (1989); and maize chlorotic mottle virus leader (MCMV), Lommel et al., (1991). See also, Della-Cioppa et al., (1987) Plant Physiol. 84:965-968. Other methods known to enhance translation can also be utilized, for example, introns, and the like.

The promoter of the expression construct will be determined by the specific requirements of the desired outcome. Generally, the target gene can be combined with promoters of choice to create increased expression of the target sequences in the tissue or organ of choice. In some instances, it may be desirable that the target gene be operably linked to a specific tissue or developmental stage of a plant. In other instances, it may be desirable to operably link the target gene to an inducible promoter for expression of the target gene only under defined inducing conditions. In other instances, it may not be desirable or required that the transcription of the target gene not be controlled as such, and in such instances a constitutive promoter may be preferred. Thus, the target gene can be combined with constitutive, tissue-specific, inducible, developmental, or other promoters for expression in plants depending upon the desired outcome.

Constitutive promoters include, for example, CaMV 35S promoter (Odell et al., 1985); rice actin (McElroy et al., 1990); maize ubiquitin (Christensen et al., 1989 and Christensen et al., 1992); pEMU (Last et al., 1991); MAS (Velten et al., 1984); and, ALS promoter (U.S. patent application Ser. No. 08/409,297). Other constitutive promoters include those taught in U.S. Pat. Nos. 5,608,149; 5,608,144; 5,604,121; 5,569,597; 5,466,785; 5,399,680; 5,268,463; and 5,608,142.

A number of inducible promoters are known in the art. For resistance genes, a pathogen-inducible promoter can be utilized. Such promoters include those from pathogenesis-related proteins (PR proteins), which are induced following infection by a pathogen; e.g., PR proteins, SAR proteins, beta-1,3-glucanase, chitinase, etc. Examples of inducible promoters, include, but are not limited to, those taught Redolfi et al. (1983) Neth. J. Plant Pathol. 89:245-254; Uknes et al. (1992) Plant Cell 4:645-656; and Van Loon (1985) Plant Mol. Virol. 4:111-116. Of particular interest are promoters that are expressed locally at or near the site of pathogen infection, including, but not limited to, those taught by Marineau et al., (1987) Plant Mol. Biol. 9:335-342; Matton et al., (1989) Molecular Plant-Microbe Interactions 2:325-331; Somsisch et al, (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93:14972-14977.

Additionally, as pathogens find entry into plants through wounds or insect damage, a wound-inducible promoter may be used in the DNA constructs of the invention. Such wound-inducible promoters include those derived from the following genes: potato proteinase inhibitor (pin II), wun1, wun2 (as taught, for example, by U.S. Pat. No. 5,428,148), win1, win2, WIP1, and MPI.

Chemical-regulated promoters can be used to modulate the expression of a gene in a plant through the application of an exogenous chemical regulator. Depending upon the objective, the promoter may be a chemical-inducible promoter, where application of the chemical induces gene expression; or a chemical-repressible promoter, where application of the chemical represses gene expression. Chemical-inducible promoters are known in the art and include, but are not limited to, the maize In2-2 promoter, which is activated by benzenesulfonamide herbicide safeners; the maize GST promoter, which is activated by hydrophobic electrophilic compounds that are used as preemergent herbicides; and the tobacco PR-1 a promoter, which is activated by salicylic acid. Other chemical regulated promoters of interest include steroid responsive promoters, such as the glucocorticoid-inducible promoter, and the tetracycline-inducible and tetracycline-repressible promoters (see, for example, U.S. Pat. Nos. 5,814,618 and 5,789,156).

Where enhanced expression in particular tissues is desired, tissue-specific promoters can be utilized. Tissue-specific promoters include those taught in Yamamoto et al. (1997) Plant J. 12(2)255-265; Kawamata et al. (1997) Plant Cell Physiol. 38(7):792-803; Hansen et al. (1997) Mol. Gen Genet. 254(3):337-343; Russel et al. (1997) Transgenic Res. 6(2):157-168; Rinehart et al. (1996) Plant Physiol. 112(3):1331-1341; Van Camp et al. (1996) Plant Physiol. 112(2):525-535; Canevasini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol. Biol. 23(6):1129-1138; Matsuoka et al. (1993) Proc Natl. Acad. Sci. USA 90(20):9586-9590; and Guevara-Garcia et al. (1993) Plant J. 4(3):495-505.

Leaf-specific promoters can similarly be used if desired, and are taught in references which include Yamamoto et al. (1997) Plant J. 12(2):255-265; Kawamata et al. (1997) Plant Cell Physiol. 38(7):792-803; Hansen et al. (1997) Mol. Gen. Genet. 254(3):337-343; Russel et al. (1997) Transgenic Res. 6(2): 157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5)773-778; Lam (1994) Results Probl. Cell Differ. 20:181-196; Orozco et al. (1993) Plant Mol. Biol. 23(6):1129-1138; Matsuoka et al. (1993) Proc. Natl. Acad. Sci USA:90(20) 9586-9590; and Guevara-Garcia et al. (1993) Plant J. 4(3):495-505.

Root-specific promoters are known and can be selected from those known to one skilled in the art and as taught in the scientific the literature. Root specific promoters, include, but are not limited to, promoters from the following genes: soybean root-specific glutamine synthetase gene; root-specific control element in the GRP 1.8 gene of French bean; mannopine synthase (MAS) gene of Agrobacterium tumefaciens; cytosolic glutamine synthetase (GS) gene expressed in roots and root nodules of soybean; hemoglobin genes from the nitrogen-fixing nonlegume Parasponia andersonii and the related non-nitrogen-fixing nonlegume Trema tomeniosa; rolC and rolD root-including genes of Agrobacterium rhizogenes; octopine synthase and TR-2′ gene; VfENOD-GRP3 gene; and, the rolB gene. Suitable promoters are also taught in U.S. Pat. Nos. 5,837,876; 5,750,386; 5,633,363; 5,459,252; 5,401,836; 5,110,732; and 5,023,179.

“Seed-preferred” promoters include both “seed-specific” promoters (those promoters active during seed development such as promoters of seed storage proteins) as well as “seed-germinating” promoters (those promoters active during seed germination). See Thompson et al. (1989) Bioassays 10:108, herein incorporated by reference. Such seed-preferred promoters include, but are not limited to, Cim1 (cytokinin-induced message); cZ10B1 (Maize 19 kDa zein); celA (cellulose synthase); gama-zein; Glob-1; bean [bgr]-phaseolin; napin; P-conglycinin; soybean lectin; cruciferin; maize 15 kDa zein; 22 kDa zein; 27 kDa zein; g-zein; waxy; shrunken 1; shrunken 2; globulin 1; etc.

In preparing expression constructs, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

In a particular embodiment, expression constructs of the present invention may comprise a chimeric target gene created by operably linking at least one peptide open reading frame to at least one target gene. The protein product of such an expression construct is a chimeric protein comprising a peptide sequence encoded by the peptide open reading frame fused to the protein of interest encoded by the gene. In one aspect, the peptide open reading frame encodes a peptide leader sequence that targets the fusion protein to a specific subcellular location within a plant. The peptide sequence may be cleaved or removed during, upon or after delivery of the fusion protein to the desired subcellular location. In one aspect, the peptide leader sequence targets the fusion protein to the mitochondria. In another aspect, the peptide leader sequence targets the fusion protein to the chloroplasts. The present invention contemplates targeting or binding of a fusion protein to any location in the cell or elsewhere for which the peptide leader sequence is specified.

Peptide sequences are taught in the art which direct proteins to specific subcellular locations. For example, a “chloroplast transit peptide” (or CTP) is the amino acid sequence that is translated in conjunction with a protein and directs the protein to the chloroplast or un-developed chloroplasts. Other transit peptides can be used to target sequences to other plastid types present in the cell in which the protein is made. “Chloroplast transit sequence” refers to a nucleotide sequence that encodes a chloroplast transit peptide. A “signal peptide” is an amino acid sequence that is translated in conjunction with a protein and directs the protein to the secretory system (Chrispeels (1991) Ann. Rev. Plant Phys. Plant Mol. Biol. 42:21-53). If the protein is to be directed to a vacuole, a vacuolar targeting signal can further be added, or if to the endoplasmic reticulum, an endoplasmic reticulum retention signal may be added. If the protein is to be directed to the nucleus, a signal peptide, if present, may be removed and a nuclear localization signal included (Raikhel (1992) Plant Phys. 100:1627-1632).

In an embodiment of the invention, a target gene may be fused to an open reading frame encoding a chloroplast transit peptide, to target the target protein to the chloroplast. Chloroplast-localized proteins may be expressed from nuclear genes as precursors and targeted to the plastid by a CTP, which is removed during the import steps. Examples of such chloroplast proteins include the small subunit of ribulose-1,5-biphosphate carboxylase (ssRUBISCO, SSU), 5-enolpyruvateshikimate-3-phosphate synthase (EPSPS), ferredoxin, ferredoxin oxidoreductase, the light-harvesting-complex protein I and protein II, and thioredoxin F. It has been demonstrated that non-plastid proteins may be targeted to the chloroplast by use of protein fusions with a CTP and that a CTP sequence is sufficient to target a protein to the plastid. Those skilled in the art will also recognize that various other chimeric constructs can be made that utilize the functionality of a particular plastid transit peptide to import a target protein into the plant cell plastid depending on the promoter tissue specificity.

Suitable examples as taught in the art include the CTP of the N-terminal transit peptide of plastocyanin for transport into chloroplasts, the N-terminal 0.26 Kb sequence derived from the Arabidopsis thaliana SSU 1a gene (SSU CTP), sedoheptulose 1,7-bisphosphatase, the small subunit of the chloroplast-localized ribulose 1,5-bisphosphate carboxylase, the modified CTP taught in U.S. Pat. No. 5,510,471, or the chloroplast phage-type RNA polymerase from Arabidopsis, RPOZ.

In other embodiments, a chimeric gene comprising the target gene and an open reading frame encoding the appropriate amino terminal peptide sequence can be constructed based upon sequence information for targeting to intracellular organelles or for secretion outside the plant cell or to the cell wall as found in naturally targeted or secreted proteins, including, for example, those taught by Klosgen et al. (1989, Mol. Gen. Genet. 217,155-161), Klosgen and Weil (1991, Mol. Gen. Genet. 225, 297-304), Neuhaus & Rogers (1998, Plant Mol. Biol. 38, 127-144), Bih et al. (1999, J. Biol. Chem. 274, 22884-22894), Morris et al. (1999, Biochem. Biophys. Res. Commun. 255, 328-333), Hesse et al. (1989, EMBO J. 8 2453-2461), Tavladoraki et al. (1998, FEBS Lett. 426, 62-66), Terashima et al. (1999, Appl. Microbiol. Biotechnol. 52, 516-523), Park et al. (1997, J. Biol. Chem. 272, 6876-6881), Shcherban et al. (1995, Proc. Natl. Acad. Sci USA 92, 9245-9249), all of which are incorporated herein by reference.

A DNA sequence differing in its codon usage but encoding the same target protein or a similar protein with substantially the same biological or functional activity, can be constructed, depending on the particular purpose. It has been described in some prokaryotic and eukaryotic expression systems that changing the codon usage to that of the host cell is desired for gene expression in foreign hosts (Bennetzen & Hall, 1982, J. Biol. Chem. 257, 3026; Itakura, 1977, Science 198, 1056-1063). Codon usage tables are available in the literature (Wada et al., 1990, Nucl. Acids Res. 18, 2367-1411; Murray et al., 1989, Nucleic Acids Research 17, 477-498) and in the major DNA sequence databases. Accordingly, synthetic DNA sequences can be constructed so that the same or substantially the same proteins are produced. In one aspect, a target gene may be optimized for expression in the transformed plant. That is, the genes can be synthesized using plant-preferred codons corresponding to the plant of interest. Methods are available in the art for synthesizing plant-preferred genes. See, for example, U.S. Pat. Nos. 5,380,831 and 5,436,391, Murray et al. (1989) Nucleic Acids Res. 17:477-498, and Perlak et al. (1991) Proc. Natl. Acad. Sci. 88:3324-3328, herein incorporated by reference.

PTGS was first discovered in transgenic plants (Baulcombe, D. C. (1996) Plant Molecular Biology 32, 79-88; and Baulcombe D. C., (2004) Nature 431: 356-363). However, related processes have been found in diverse eukaryotic organisms, including filamentous fungi, nematodes, and a variety of animal systems where it is referred to as RNA interference (see, for example, McManus M. T., and Sharp P. A (2002) Nature Reviews Genetics 3: 737-747; and, Dykxhoorn D. M. et al. (2003) Nature Reviews Molecular Cell Biology 4: 457-467). Double-stranded RNA (dsRNA) induces PTGS in many systems (Montgomery and Fire, 1998; Wianny and Zernicka-Goetz, 2000; Waterhouse and Graham, 1998) and, in plants, it can also be triggered by cytoplasmically replicating viruses, many of which produce dsRNA replication intermediates (Kumagai et al., 1995; Ratcliff et al., 1997). Once the mechanism is activated, homologous RNA is degraded, whether it is transcribed from the transgene, the endogenous gene, or the viral RNA. The observation that plant viruses can act both as inducers and as targets of PTGS has led to the idea that PTGS evolved as a defense mechanism against viruses in plants (Kumagai et al., 1995; Baulcombe, 1999). A number of conserved gene products have been implicated as being required or important for PTGS to function in suppressing gene silencing.

It has been previously reported that certain plant viruses encode proteins that can suppress PTGS (see for example Anandalaksmi, et al., 1998, Beclin et al., 1998, Brigneti et al., 1998, Kasschau and Carrington, 1998, and Voinnet, et al., 1999, 2000). In one aspect, the PTGS suppressor constructs of the present invention comprise at least one promoter operably linked to at least one gene encoding a viral or non-viral protein that suppress one or more components of PTGS. In particular, the PTGS suppressor constructs of the present invention comprise gene, and gene product thereof, for the helper component-protease (P1/HC-Pro) protein derived from plant potyviruses (Anandalakshmi et al., 1998; Kasschau and Carrington, 1998). In one aspect, the PTGS suppressor constructs of the present invention comprise the P1/HC-Pro protein gene, and gene product thereof, derived from tobacco etch virus. In another aspect, the PTGS suppressor constructs further comprise variants of P1/HC-Pro, in which a nine-nucleotide insertion has been introduced into the construct at the junction of P1 and HC-Pro. In another aspect, the PTGS suppressor constructs comprise P1/HC-Pro, and its variants, derived from tobacco etch virus (TEV).

Suitable forms of P1/HC-Pro have been described and are incorporated herein in their entirety (Mallory et al., 2001, Mallory et al., 2002, and U.S. Pat. Nos. 6,395,962 and 6,753,139, and United States Patent Application Publication No. 2004/0268441 A1). Other forms and variants of P1/HC-Pro are known to one skilled in the art and are within the scope and spirit of the present invention. In one aspect, the PTGS suppressor constructs comprise nucleic acid suppression of one or more components of PTGS, including RNAi suppression of one or more components of PTGS.

The PTGS suppressor constructs of the present invention may further comprise a gene encoding a protein that acts to boost or enhance the function of another PTGS suppressor gene product, including, for example, P1/HC-Pro. A suitable protein that enhances the activity of P1/HC-Pro is a calmodulin-like polypeptide named rgs-CaM as described in U.S. Patent Application Publication 2005/0022262 A1, which is incorporated herein by reference in its entirety.

In a further aspect, the PTGS suppressor constructs of the present invention may comprise the 2b gene of cucumber mosaic virus, and the gene product thereof (Brigneti et al., 1998). In one aspect, the PTGS suppressor constructs may also comprise the following genes, and gene products thereof: the P19 gene from tomato bushy stunt virus Voinnet et al., 1999, the AC2 genes of geminiviruses (Voinnet et al., 1999), and the P1 gene of rice yellow mottle virus (Voinnet et al., 1999).

In one aspect, the methods and compositions of the present invention comprise a PTGS suppressor construct comprising a gene encoding P1/HC-Pro derived from TEV and a viral-based vector.

Target genes of the present invention may be derived from plant, animal, microbial or viral sources. Target genes may further comprise genes that are in whole or in part synthetic in origin and not derived from any natural source. Target genes comprise those encoding agronomic traits, insect resistance, disease resistance, herbicide resistance, sterility, grain characteristics, and the like. The target genes of the present invention may be involved in metabolism of oil, starch, carbohydrates, and nutrients. Target genes include, but are not limited to, those conferring environmental- or stress-resistance traits, disease-resistance traits, and traits affecting agronomic performance. Target genes may also comprise those genes encoding proteins important for the synthesis of proteins, peptides, fatty acids, lipids, waxes, oils, starches, sugars, carbohydrates, flavors, odors, toxins, carotenoids, hormones, polymers, flavonoids, storage proteins, phenolic acids, alkaloids, lignins, tannins, celluloses, glycoproteins, and glycolipids.

Target genes of the present invention may comprise genes encoding proteins of value as pharmaceuticals or diagnostic reagents, including, but not limited to, vaccine proteins, antibodies, enzymes, receptor antagonists, receptor agonists, soluble receptors, anti-microbial peptides, and therapeutic proteins. Target genes of the present invention may further comprise proteins that are of value as feedstocks or reagents for industry, including, but not limited to, industrial enzymes, anti-microbial peptides, and the like.

In one embodiment, the methods and compositions of the present invention can be used to produce peptides or proteins that cannot effectively be commercially produced by existing gene expression systems. For example, some proteins cannot be expressed in conventional bacterial, fungal, or mammalian expression systems because the protein interferes with cell viability, cell proliferation, cellular differentiation, or protein assembly in mammalian cells. In other cases, although the protein of interest can be successfully expressed in conventional expression systems, the cost makes use of such systems prohibitive or not commercially viable. Such proteins include, but are not limited to, retinoblastoma protein, p53, angiostatin, and leptin. Likewise, the methods of the invention can be used to produce mammalian regulatory proteins. Other sequences of interest include proteins, hormones, growth factors, cytokines, including for example, insulin, growth hormone, particularly human growth hormone, interferon, particularly α-interferon, β-glucocerebrosidase, serum albumin, particularly human serum albumin, hemoglobin, and collagen.

In one embodiment, the methods may be used to express recombinant target proteins that are encoded by disease and insect resistance genes in the plant. In another embodiment, the methods of the invention can be used to produce recombinant seed products. Such seed proteins of interest include, but are not limited to, storage proteins and proteins with enhanced nutritional value.

Generally, the methods of the invention can be used for the expression of any gene encoding a recombinant target protein of interest, including therapeutic or immunogenic peptides and proteins, genes to reproduce enzymatic pathways for chemical synthesis, genes to enhance an enzymatic pathway for enhanced expression of a particular intermediate or final product, industrial processes, and the like.

The methods and compositions of the present invention are particularly well-suited for the simultaneous expression of two or more recombinant target proteins. In one aspect, the methods and compositions of the present invention may be used to express and accumulate two or more different proteins, wherein the functions are unrelated. Using this system, such proteins could be produced in the same biomass, and subsequently extracted and separated, so that each forms a separate product stream.

Many proteins of commercial value are multimeric complexes of two or more distinct protein subunits. In another aspect, the methods and compositions of the present invention may be used to introduce into plants simultaneously multiple target protein expression constructs, wherein each target protein expression construct carries a gene for a distinct subunit of a multimeric protein complex. In one embodiment, the methods and compositions of the present invention comprise expression of an antibody of therapeutic, diagnostic or other commercial interest wherein the heavy and light chains of the antibody are each encoded on distinct target protein expression constructs. In a further embodiment, the methods and compositions of the present invention are used to express chimeric human-mouse antibodies.

In another aspect, the methods and compositions of the present invention may be used for the introduction of multiple target protein expression constructs into plants, wherein there is a particular advantage to the expression of two or more target proteins. For example, it may be valuable to introduce into a plant two or more target protein expression constructs wherein each two or more insect and disease resistance genes are simultaneously introduced into the subject plant. In another example, it may be valuable to simultaneously introduce into a plant a multitude of recombinant target proteins wherein each such protein has a particular value or attribute such as increased disease resistance, acceleration of seed maturation, and increased production of specific seed products.

The target protein expression constructs and PTGS suppressor constructs of the present invention may be introduced into plant cells by any means of plant transformation generally known to one skilled in the art. Plants transformed with a target protein expression construct, PTGS expression construct or both types of construct of the present invention may be produced by standard techniques known in the art. For example, DNA can be transformed into plant cells using any suitable technology, such as a disarmed Ti-plasmid vector carried by Agrobacterium exploiting its natural gene transferability (as taught, for example, in EP-A-270355; EP-A-0116718; and, U.S. Pat. No. 5,563,055); particle or microprojectile bombardment (as taught, for example, in U.S. Pat. Nos. 4,945,050 and 5,100,792; EP-A444882; and, EP-A434616); microinjection (as taught, for example, in WO 92/09696; WO 94/00583; EP 331083; EP 175966); other forms of direct DNA uptake (as taught, for example, in DE 4005152; WO 9012096; U.S. Pat. No. 4,684,611); liposome-mediated DNA uptake; or the vortexing method (see, for example, Kindle (1990) Proc. Nat. Acad. Sci USA 87:1228).

Agrobacterium transformation is widely used by those skilled in the art to transform dicotyledonous species. However, Agrobacterium mediated transformation is now emerging as a highly efficient transformation method in monocots, as taught, for example in Hiei, et al. (1994) The Plant Journal 6:271-282); Shimamoto, K. (1994) Current Opinion in Biotechnology 5:158-162; Vasil, et al. (1992) Bio/Technology 10:667-674; and, Vasil et al. (1996) Nature Biotechnology 14:702.

Microprojectile bombardment, electroporation, and direct DNA uptake are preferred in plant species and/or plant tissues for which Agrobacterium is inefficient or ineffective. Alternatively, a combination of different techniques may be employed to enhance the efficiency of the transformation process, e.g., bombardment with Agrobacterium-coated microparticles (as taught, for example, in EP-A-486234) or microprojectile bombardment to induce wounding followed by co-cultivation with Agrobacterium (as taught, for example, in EP-A-486233).

The present invention further comprises novel methods suitable for the bulk transformation of plants, which is also referred to herein as “bulk agroinfiltration”. The bulk agroinfiltration methods of the present invention can be used to transform plants grown in greenhouses, plots, and commercial acreage. In general, the bulk agroinfiltration methods of the present invention comprise contacting a suspension of recombinant Agrobacterium into leaves and other exposed areas of the subject plant. Although this can be accomplished with little if any damage to the exposed tissues, in some cases transient damage of the leaves or other exposed tissues is desirable to enhance the infection. In all cases, the plant remains healthy and viable, even if the surface of the leaves and exposed areas of the plant are physically perturbed sufficiently to allow transformation by Agrobacterium that is sprayed, washed, or aerosolized onto, at or near the leaves and exposed plant surfaces. In one embodiment, a mixture of Agrobacterium containing one or more desired target protein expression constructs and a PTGS suppressor construct are grown to mid-log phase, then resuspended to an optical density (OD₆₀₀) of 1.0, and then sprayed onto the damaged leaves and exposed areas of the subject plant. In one aspect, the subject plant is Nicotiana tabacum. In another embodiment, a mixture of Agrobacterium containing one or more desired target protein expression constructs are grown to mid-log phase, resuspended to an optical density of about 1.0, and then sprayed onto the leaves and exposed areas of the subject plant. In another aspect, the subject plant is a transgenic Nicotiana tabacum stably expressing a PTGS suppressor.

The choice of a transformation technology will be determined by its efficiency to transform certain plant species as well as the experience and preference of the person practicing the invention with a methodology of choice. It will be apparent to the skilled person that the choice of a transformation system to introduce nucleic acid into plant cells is not essential to or a limitation of the invention.

By “variants” in the context of nucleic acids or proteins is intended to mean substantially similar sequences. For example, for nucleotide sequences, conservative variants include those sequences that, because of the degeneracy of the genetic code, encode the amino acid sequence of the booster of the invention. Variant nucleotide sequences include synthetically derived sequences, such as those generated for example, using site-directed mutagenesis. Generally, nucleotide sequence variants of the invention will have at least 40%, 50%, 60%, 70%, 80%, 85%, 90%, and up to 95% or more sequence identity to its respective native nucleotide sequence. Thus, some fragments may also be variants.

“Variant” in the context of proteins is intended to mean a protein derived from the native protein by deletion or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or human manipulation. Conservative amino acid substitutions will generally result in variants that retain biological function. Variant proteins that retain a desired biological activity are encompassed within the subject invention. Variant proteins of the invention may include those that are altered in various ways including amino acid substitutions, deletions, truncations, and insertions. Methods for such manipulation are generally known in the art. See, for example, Kunkel (1985) Proc. Natl. Acad. Sci. USA 82:488-492; Kunkel et al. (1987) Methods and Enzymol; 154:367-382; and the references cited therein.

Sequence relationships between two or more nucleic acids or polynucleotides are generally defined as sequence identity, percentage of sequence identity, and substantial identity. In determining sequence identity, a “reference sequence” is used as a basis for sequence comparison. The reference may be a subset or the entirety of a specified sequence. That is, the reference sequence may be a full-length gene sequence or a segment of the gene sequence.

Methods for alignment of sequences for comparison are well known in the art. See, for example, Smith et al. (1981) Adv. Appl. Math. 2:482; Needleman et al. (1970) J. Mol. Biol. 48:443; Pearson et al. (1988) Proc. Natl. Acad. Sci. 85:2444; CLUSTAL in the PC/Gene Program by Intelligenetics, Mountain View, Calif.; GAP, BESTFIT, BLAST, FASTA, and TFASTA in the Wisconsin Genetics Software Package, Genetics Computer Group (GCG), 575 Science Drive, Madison, Wis., USA. Preferred computer alignment methods also include the BLASTP, BLASTN, and BLASTX algorithms. See also, Altschul et al. (1990) J. Mol. Biol. 215:403-410.

“Sequence identity” or “identity” in the context of nucleic acid or polypeptide sequences refers to the nucleic acid bases or residues in the two sequences that are the same when aligned for maximum correspondence over a specified comparison window. “Percentage of sequence identity” refers to the value determined by comparing two optimally aligned sequences over a comparison window, wherein the portion of the polynucleotide sequence in the comparison window may comprise additions or deletions as compared to the reference window for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Polynucleotide sequences having “substantial identity” are those sequences having at least about 50%-60%, 60%-70%, 70%-80%, 80%-90%, at least 90%, and at least 95%, compared to a reference sequence using one of the alignment programs described above. Preferably sequence identity is determined using the default parameters determined by the program. Substantial identity of amino acid sequence generally means sequence identity of at least 50%, more preferably at least 70%, 80%, 90%, and most preferably at least 95%.

As indicated, fragments and variants of the nucleotide sequences of the invention are encompassed herein. By “fragment” is intended a portion of the nucleotide sequence. Fragments may be generated by a number of methods well known in the art, such as by use of commercially available restriction enzymes, exonucleases such as Bal31, or by chemical synthesis. Fragments of the booster sequence will generally encode polypeptides which retain one or more of the biological activities of the native protein. Activities can be tested and confirmed by following examples set forth herein. Alternatively, fragments of the polynucleotide sequences may or may not retain biological activity. Such sequences may be useful as hybridization probes, as antisense constructs, or as co-suppression sequences. Thus, fragments of a nucleotide sequence may range from at least about 4-20 nucleotides, about 50 nucleotides, about 100 nucleotides, and up to the full-length nucleotide sequence of the particular polynucleotide of interest.

The present invention may be used for transformation of any plant species, including, but not limited to, corn (Zea mays), canola (Brassica napus, Brassica rapa ssp.), alfalfa (Medicago sativa), rice (Oryza sativa), rye (Secaie cereale), sorghum (Sorghum bicolor, Sorghum vulgare), sunflower (Helianthus annuus), wheat (Triticum aestivum), soybean (Glycine max), tobacco (Nicotiana tabacum), potato (Solanum tuberosum), peanuts (Arachis hypogaea), cotton (Gossypium hirsutum), sweet potato (Ipomoea batatus), cassaya (Manihot esculenta), coffee (Cofea ssp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidental), macadamia (Macadamia integrifolia), almond (Prunus amygdalus), sugar beets (Beta vulgaris), oats, barley, vegetables, ornamentals, and conifers.

Plants of the present invention are crop plants, which include, but are not limited, tobacco, cereals and pulses, maize, wheat, potatoes, tapioca, rice, sorghum, millet, cassava, barley, pea, and other root, tuber, or seed crops. Important seed crops are oil-seed rape, sugar beet, maize, sunflower, soybean, and sorghum. Horticultural plants to which the present invention may be applied may include lettuce; endive; and vegetable brassicas including cabbage, broccoli, and cauliflower; and carnations and geraniums. The present invention may be applied to tobacco, cucurbits, carrot, strawberry, sunflower, tomato, pepper, chrysanthemum, poplar, eucalyptus, and pine.

Grain plants that provide seeds of interest include oil-seed plants and leguminous plants. Seeds of interest include grain seeds, such as corn, wheat, barley, rice, sorghum, rye, etc. Oil seed plants include cotton, soybean, safflower, sunflower, Brassica, maize, alfalfa, palm, coconut, etc. Leguminous plants include beans and peas. Beans including guar, locust bean, fenugreek, soybean, garden beans, cowpea, mungbean, lima bean, fava bean, lentils, chickpea, etc.

In one embodiment, the compositions of the present invention comprise a target protein expression construct comprising a plasmid wherein the target gene is operably linked to the 3′ end of a DNA sequence encoding a CTP derived from the small subunit of RuBisCO from tobacco. Expression of such a chimeric target protein results in the production of a target protein of interest linked at it's amino terminus to the CTP. The chimeric target protein is transported into the chloroplast. While not wishing to be bound by a particular theory, it is believed that the CTP sequence is removed from the target protein of interest at the time of importation of the chimeric target protein into the chloroplast. The target protein of interest accumulates in the chloroplast, where it is protected from cytosolic proteases that could degrade the protein. Because the target protein expression construct is introduced into a plant which also carries the P1/HC-Pro element, the effect is a dual protection of the desired product. The transcript is protected from degradation by PTGS, and the desired protein product is protected against degradation by cytosolic proteases.

In an embodiment, the methods and compositions of the present invention comprise infection of a recipient plant with a target protein expression construct which carried out by infusing the leaves of recipient plants with A. tumefaciens carrying a modified Ti plasmid incorporating a target gene of interest operably linked to the 3′ end of a DNA sequence encoding the CTP derived from the small subunit of RuBisCO as described above. The recipient plant can be a tobacco plant that is stably transformed with the P1/HC-Pro element. Transient expression of the target protein gene occurs, and replicating transcripts are produced in the plant cells. One benefit of this process is that stable transgenic plants carrying the target protein expression construct do not have to be made in advance which saves both time and labor. Thus, the present invention allows decisions about which proteins are to be expressed to be delayed to more accurately predict the needs of the consumer. In this embodiment, expression of the target protein or proteins occurs in the treated plants within at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30 days following infection of the target plant with Agrobacterium. In another embodiment, peak expression of the target protein or proteins occurs in the period of 1-3, 2-4, 3-5, 4-6, 5-7, 6-8, 7-9, 8-10, 9-11, 10-12, 11-13, 12-14, 13-15, 14-16, 15-17, 16-18, 17-19, 20-22, 21-23, 22-24, 23-25, 24-26, 25-27, 26-28, 27-29, 28-30, and 29-31 days following infection with of the target plant with Agrobacterium. In a further embodiment, peak expression of the target protein or proteins occurs in the period 4-11, 5-12, 6-13, 7-14, 8-15, 9-16, 10-17, and 11-18 days following infection of the target plant with Agrobacterium.

In another embodiment, the methods and compositions of the present invention comprise plants that are co-infected with Agrobacterium comprising a target protein expression construct and Agrobacterium comprising a PTGS suppressor construct comprising a P1/HC-Pro gene or suitable variant thereof. This procedure is useful for at least two reasons. First, it circumvents the need to produce any stably transformed plants at any time, saving substantial amounts of time and labor. Secondly, this procedure allows the use of any tobacco genotype as the recipient stock, facilitating use of the most appropriate tobacco line for a specific application. Thus, it is possible to produce numerous Agrobacterium cultures, each of which contains a different amplicon construction encoding a different protein. Using the procedures described above, it is possible to infect wild-type tobacco with a complex mixture of cultures, one carrying the P1/HC-Pro coding sequence, the remainder carrying constructs encoding an array of different proteins, each fused with a chloroplast transit peptide. In this way, it is possible to cause the recipient tobacco plant to produce several different proteins simultaneously, each of which might then be isolated from the plant tissue. The advantage of this procedure is that it would allow production of numerous proteins in the shortest possible time, using the minimum amount of space and other resources. This might be especially advantageous when the tobacco is produced in a greenhouse for containment purposes, since it would permit recovery of several valuable products from the same biomass, thus increasing product value relative to input cost.

In one embodiment, the present invention comprises methods to enrich the crude extract for the protein of interest in order to facilitate further purification steps. For example, the desired protein in certain embodiments is sequestered in the chloroplast when the amplicon construction includes a CTP sequence, it is possible to perform a preliminary concentration of the protein by isolating intact chloroplasts prior to lysing them to obtain the protein. This increases the effective concentration of protein available for subsequent purification steps. Because protein recovery is often improved by keeping protein concentration high, this improves overall yield of the desired protein. Further, this procedure may insure that the desired protein has maximal protection from proteolysis afforded by chloroplast sequestration.

The methods and compositions of the present invention also comprise methods for plant management to maximize yields of the target protein. Yield of target protein in using the methods and compositions taught by the present invention maximize the interaction of several factors. These include the developmental stage reached by the recipient tobacco plant prior to inoculation with Agrobacterium, length of time between inoculation and harvest of biomass for protein extraction, and various growing conditions (temperature, light, fertility, day-length, etc.) under which the inoculated plants are maintained.

Methods of the present invention comprise the expression of useful recombinant proteins (e.g., prophylactic or therapeutic vaccines, antibodies, enzymes, etc.) in tobacco at higher levels than is typically possible using more standard transgenic plant technologies. Further, as described herein, recombinant proteins may be protected from degradation by cytosolic proteases by sequestering the recombinant protein in specific subcellular locations, such as inside chloroplasts. The methods and compositions of the present invention are amenable to use in a wide variety of plants, including, but not limited to, tobacco. Generally, the methods and compositions of the present invention may be used with any plant in which a viral replicon can be replicated.

In one aspect, the present invention comprises a method for producing a polypeptide in a plant, including contacting the plant with a recombinant composition, wherein the recombinant composition comprises a recombinant amplicon comprising a nucleic acid encoding at least one transit peptide or at least one signal peptide operably linked to a nucleic acid encoding the polypeptide. In one embodiment, the recombinant composition further comprises a recombinant bacterial vehicle (e.g., Agrobacterium). The Agrobacterium may be introduced in a leave of the plant by the application of pressure or vacuum. The Agrobacterium may also be sprayed onto the plant. In one embodiment, a wound may be formed in the plant before spraying the Agrobacterium onto the plant. In another embodiment, the plant is a transgenic tobacco plant TEV-B. In yet another embodiment, the recombinant amplicon is a recombinant potato virus X amplicon. In still another embodiment, the at least one transit peptide is a chloroplast-targeting transit peptide. In addition, the at least one signal peptide may comprise an N-terminal signal peptide, and wherein the presence of the N-terminal signal peptide results in the secretion of the polypeptide into an apoplast in a plant cell. In another embodiment, the at least one signal peptide may comprise an N-terminal signal peptide and a C-terminal signal peptide, and wherein the presence of the N-terminal signal peptide and the C-terminal signal peptide results in the retention of the polypeptide in an endoplasmic reticulum in a plant cell. In yet another embodiment, the recombinant composition further comprises a nucleic acid encoding a suppressor of a post transcriptional gene silencing process. The suppressor of a post transcriptional gene silencing process may be a suppressor of virus or plant origin, such as, P1/HC-Pro or a variant thereof. In one embodiment, the plant, e.g., a tobacco plant (including, such as, N. tabacum and N. benthamiana), may comprise a nucleic acid encoding a suppressor of a post transcriptional gene silencing process.

In another aspect, the present invention comprises a polypeptide produced in accordance with a method comprising contacting a plant with a recombinant composition, wherein the recombinant composition comprises a recombinant amplicon comprising a nucleic acid encoding at least one transit peptide or at least one signal peptide operably linked to a nucleic acid encoding the polypeptide. In one embodiment, the recombinant composition further comprises a recombinant bacterial vehicle (e.g., Agrobacterium). The Agrobacterium may be introduced in a leave of the plant by the application of pressure or vacuum. The Agrobacterium may also be sprayed onto the plant. In one embodiment, a wound may be formed in the plant before spraying the Agrobacterium onto the plant. In another embodiment, the plant is a transgenic tobacco plant TEV-B. In yet another embodiment, the recombinant amplicon is a recombinant potato virus X amplicon. In still another embodiment, the at least one transit peptide is a chloroplast-targeting transit peptide. In addition, the at least one signal peptide may comprise an N-terminal signal peptide, and wherein the presence of the N-terminal signal peptide results in the secretion of the polypeptide into an apoplast in a plant cell. In another embodiment, the at least one signal peptide may comprise an N-terminal signal peptide and a C-terminal signal peptide, and wherein the presence of the N-terminal signal peptide and the C-terminal signal peptide results in the retention of the polypeptide in an endoplasmic reticulum in a plant cell. In yet another embodiment, the recombinant composition further comprises a nucleic acid encoding a suppressor of a post transcriptional gene silencing process. The suppressor of a post transcriptional gene silencing process may be a suppressor of virus or plant origin, such as, P1/HC-Pro or a variant thereof. In one embodiment, the plant, e.g., a tobacco plant (including, such as, N. tabacum and N. benthamiana), may comprise a nucleic acid encoding a suppressor of a post transcriptional gene silencing process.

In yet another aspect, the present invention comprises a plant comprising a recombinant composition, wherein the recombinant composition comprises a recombinant amplicon comprising a nucleic acid encoding at least one transit peptide or at least one signal peptide operably linked to a nucleic acid encoding the polypeptide. In one embodiment, the recombinant composition further comprises a recombinant bacterial vehicle (e.g., Agrobacterium). The Agrobacterium may be introduced in a leave of the plant by the application of pressure or vacuum. The Agrobacterium may also be sprayed onto the plant. In one embodiment, a wound may be formed in the plant before spraying the Agrobacterium onto the plant. In another embodiment, the plant is a transgenic tobacco plant TEV-B. In yet another embodiment, the recombinant amplicon is a recombinant potato virus X amplicon. In still another embodiment, the at least one transit peptide is a chloroplast-targeting transit peptide. In addition, the at least one signal peptide may comprise an N-terminal signal peptide, and wherein the presence of the N-terminal signal peptide results in the secretion of the polypeptide into an apoplast in a plant cell. In another embodiment, the at least one signal peptide may comprise an N-terminal signal peptide and a C-terminal signal peptide, and wherein the presence of the N-terminal signal peptide and the C-terminal signal peptide results in the retention of the polypeptide in an endoplasmic reticulum in a plant cell. In yet another embodiment, the recombinant composition further comprises a nucleic acid encoding a suppressor of a post transcriptional gene silencing process. The suppressor of a post transcriptional gene silencing process may be a suppressor of virus or plant origin, such as, P1/HC-Pro or a variant thereof. In one embodiment, the plant, e.g., a tobacco plant (including, such as, N. tabacum and N. benthamiana), may comprise a nucleic acid encoding a suppressor of a post transcriptional gene silencing process.

In still another aspect, the present invention comprises a plant cell comprising a recombinant composition, wherein the recombinant composition comprises a recombinant amplicon comprising a nucleic acid encoding at least one transit peptide or at least one signal peptide operably linked to a nucleic acid encoding the polypeptide. In one embodiment, the plant is a transgenic tobacco plant TEV-B. In another embodiment, the recombinant amplicon is a recombinant potato virus X amplicon. In yet another embodiment, the at least one transit peptide is a chloroplast-targeting transit peptide. In addition, the at least one signal peptide may comprise an N-terminal signal peptide, and wherein the presence of the N-terminal signal peptide results in the secretion of the polypeptide into an apoplast in a plant cell. In another embodiment, the at least one signal peptide may comprise an N-terminal signal peptide and a C-terminal signal peptide, and wherein the presence of the N-terminal signal peptide and the C-terminal signal peptide results in the retention of the polypeptide in an endoplasmic reticulum in a plant cell. In yet another embodiment, the recombinant composition further comprises a nucleic acid encoding a suppressor of a post transcriptional gene silencing process. The suppressor of a post transcriptional gene silencing process may be a suppressor of virus or plant origin, such as, P1/HC-Pro or a variant thereof. In one embodiment, the plant cell, e.g., a tobacco plant cell (including, such as, N. tabacum and N. benthamiana), may comprise a nucleic acid encoding a suppressor of a post transcriptional gene silencing process.

Also provided is an isolated nucleic acid comprising a recombinant amplicon, which comprises a nucleic acid encoding at least one transit peptide or at least one signal peptide operably linked to a nucleic acid encoding the polypeptide. In one embodiment, the recombinant amplicon is a recombinant potato virus X amplicon. In another embodiment, the at least one transit peptide is a chloroplast-targeting transit peptide. In yet another embodiment, the chloroplast-targeting transit peptide comprises at least one of amino acid sequences of SEQ ID NOs. 2, and 16-22. In addition, the at least one signal peptide may comprise an N-terminal signal peptide, and wherein the presence of the N-terminal signal peptide results in the secretion of the polypeptide into an apoplast in a plant cell. In another embodiment, the at least one signal peptide may comprise an N-terminal signal peptide and a C-terminal signal peptide, and wherein the presence of the N-terminal signal peptide and the C-terminal signal peptide results in the retention of the polypeptide in an endoplasmic reticulum in a plant cell. In yet another embodiment, the isolated nucleic acid further comprises a nucleic acid encoding a suppressor of a post transcriptional gene silencing process. The suppressor of a post transcriptional gene silencing process may be a suppressor of virus or plant origin, such as, P1/HC-Pro or a variant thereof.

The foregoing description includes the best presently contemplated mode of carrying out the present invention. This description is made for the purpose of illustrating the general principles of the present inventions and should not be taken in a limiting sense. This invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention.

All terms used herein are considered to be interpreted in their normally accepted usage by those skilled in the art. Patent and patent applications or references cited herein are all incorporated by reference in their entireties.

### EXAMPLES

High-level expression of transgenes is essential for cost-effective production of valuable pharmaceutical proteins in plants. However, transgenic proteins often accumulate in plants at low levels. Low levels of protein accumulation can be caused by many factors including post-transcriptional gene silencing (PTGS) and/or rapid turnover of the transgenic proteins. A novel Amplicon-plus Targeting Technology (APTT) that overcomes both of these factors is disclosed. By using this technology, the highly-labile L1 protein of canine oral papillomavirus (COPV L1) is expressed by infecting transgenic tobacco plants expressing a suppressor of PTGS with a PVX amplicon carrying a gene encoding L1, and targeting the vaccine protein into the chloroplasts. Further, a scalable “wound-and-agrospray” inoculation method is also disclosed, which permits high-throughput Agrobacterium inoculation of N. tabacum, and a spray-only method (named “agrospray”) for use with N. benthamiana to allow large-scale application of this technology. The good yield and short interval from inoculation to harvest characteristic of APTT, combined with the potential for high-throughput achieved by use of the agrospray inoculation protocol, make this system a very promising technology for producing high value recombinant proteins, especially those known to be highly labile, in plants for a wide range of applications including producing vaccines against rapidly evolving pathogens and for the rapid response needed to meet bio-defense emergencies.

### Example 1

### Expression Vectors

The L1 gene from canine oral papillomavirus (COPV) (Suzich et al., 1995), which encodes the primary capsid protein L1, was modified to use plant-preferred codons, and to eliminate potential cryptic splicing sites and polyadenylation signal-like internal sequences (Perlak et al., 1991) by Aptagen, Inc, Herndon, Va. The custom-synthesized version of the COPV L1 gene was designated “vcp” (GenBank accession no. DQ508357).

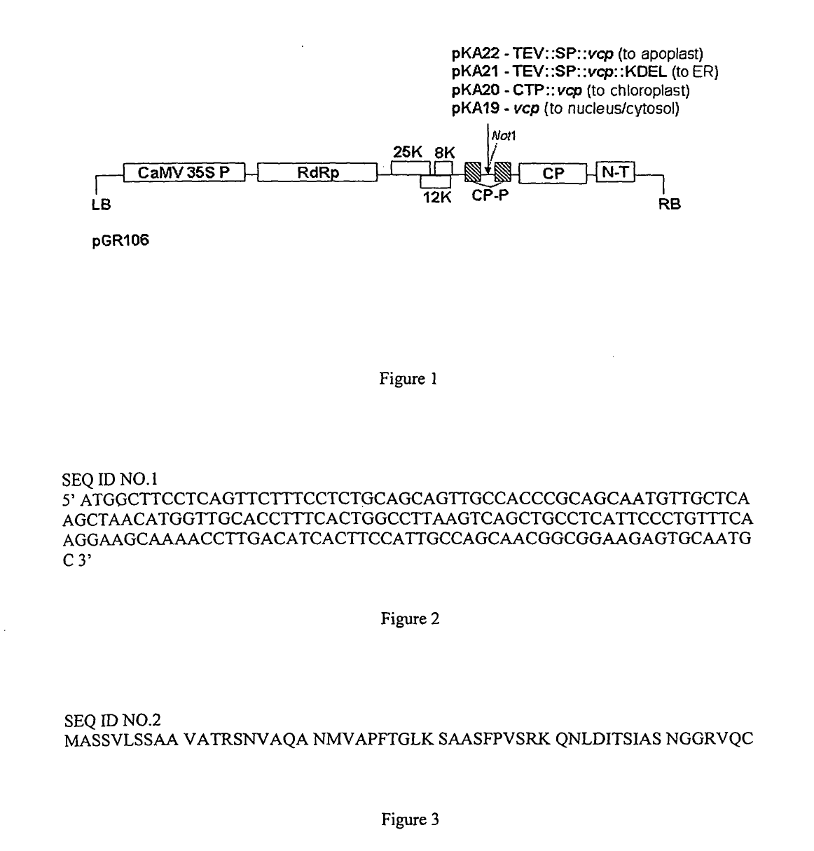

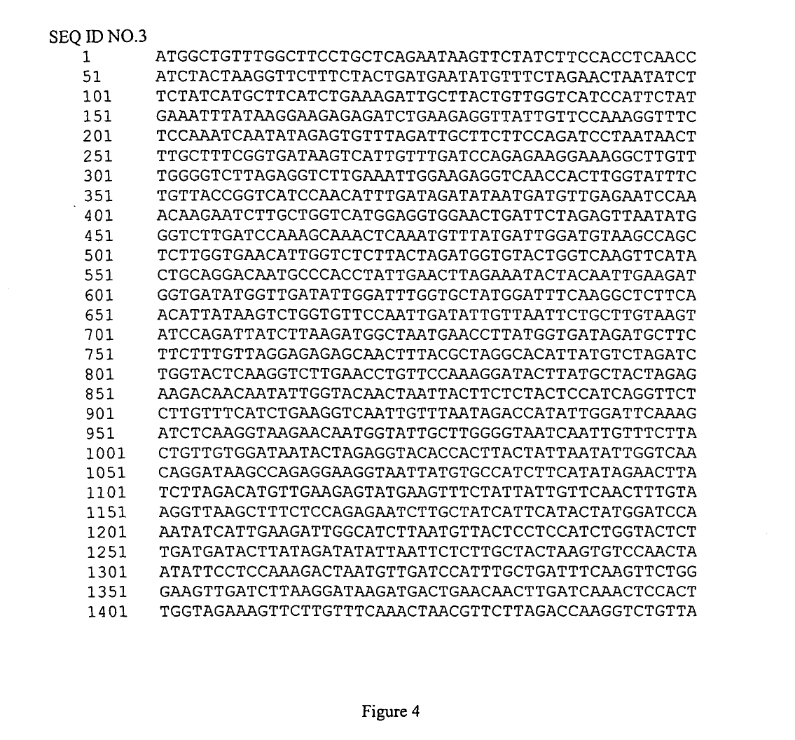

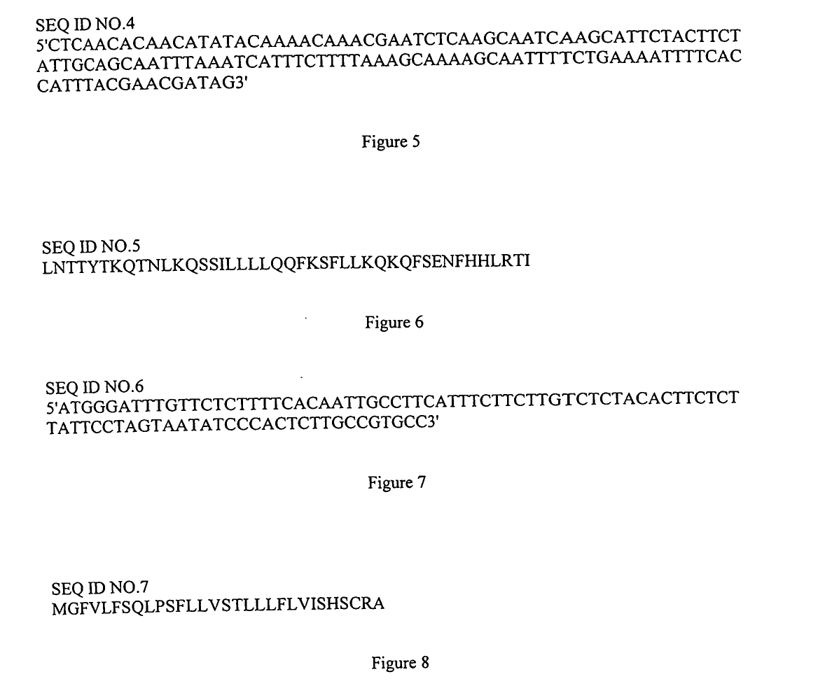

Four constructs based on the PVX amplicon vector pGR106 (Lu et al., 2003) were made to express the L1 protein in cytosol, and to target the protein to the chloroplast, the endoplasmic reticulum (ER), or the apoplast, respectively (FIG. 1). The vcp gene was inserted at the blunted NotI site in pGR106 to make “pKA19” (FIG. 1). For chloroplast targeting, the chloroplast transit peptide (CTP) coding sequence of ribulose-1,5-bisphosphate carboxylase oxidase (rubisco) small subunit from tobacco (GenBank accession no. X02353) was obtained by PCR amplification of the sequence from tobacco DNA. The sequence was then fused with the 5′ terminus of the vcp coding sequence. PCR based precise in-frame fusion of the CTP and the vcp gene was carried out using the three primer approach (Yon and Fried, 1989). The resulting fusion gene (CTP::vcp, 1.7 kb) was inserted into vector pGR106 at the blunted NotI site and the resulting construct was designated “pKA20” (FIG. 1). In a similar approach, the TEV 5′ untranslated leader sequence (for translation enhancement) and the ER-targeting signal peptide (SP) from the tobacco PR1a gene (Xu et al., 2002) was fused in-frame with the N-terminus of the vcp coding sequence. The sequence encoding the endoplasmic reticulum retrieval signal KDEL was included as a part of the reverse PCR primer so that it was fused in frame with the 3′ terminus of the vcp gene. The resulting fusion sequence (TEV leader::SP::vcp::KDEL) was inserted at the blunted NotI site in vector pGR106, resulting in the construct designated “pKA21” (FIG. 1). A similar construct was made without the KDEL to target the protein to the apoplast. The fusion construct (TEV leader::SP::vcp), cloned in pGR106 as described above, was designated “pKA22” (FIG. 1). These constructs were used to transform Agrobacterium tumefaciens strain containing the helper plasmid pJIC SA_Rep using the freeze-thaw method (An et al., 1988). In FIG. 1, using the pGR106 vector base, “CaMV 35S P” is the CaMV 35S promoter; “RdRp” is the sequence encoding RNA-dependent RNA polymerase from PVX; “25K”, “12K”, and “8K” are, collectively, the “triple gene block” encoding three specific movement proteins of PVX; “CP-P” designates a duplicated coat protein subgenomic promoter from PVX; “CP” designates a sequence encoding the PVX coat protein; and, “N-T” designates the nos terminator. “LB” and “RB” designate the left and right border sequences of the T-DNA of Agrobacterium tumefaciens. The codon optimized vcp (a sequence encoding the L1 coat protein of canine oral papilomavirus, optimized for use in plants) cDNA and its derivatives were inserted at the NotI site of a polylinker within pGR106, driven by a PVX CP subgenomic promoter.

### Example 2

### Agroinfiltration of Tobacco Plants

Experiments were carried out using Nicotiana tabacum cv. Xanthi, N. benthamiana, and homozygous transgenic TEV-B plants (made in the tobacco cv. Xanthi) containing a modified version of the P1/HC-Pro gene from TEV that suppresses post-transcriptional gene silencing, but which does not cause deleterious changes in the plant phenotype associated with the wild-type version of P1/HC-Pro. Preparation of Agrobacterium cultures and infiltration of tobacco plants were carried out as described (English et al., 1997) with the following modifications. The recombinant bacteria were grown overnight in 50 ml of LB medium containing 100 μM acetosyringone and 10 μM MES (pH 5.6), and subsequently were pelleted by centrifugation at 4000 g for 5 min. The pellets were resuspended in the infection medium (Murashige and Skoog salts with vitamins, 2% sucrose, 500 μM MES (pH 5.6), 10 μM MgSO₄, and 100 μM acetosyringone) to OD₆₀₀=0.4-1.0 and subsequently held at 28° C. for 2-3 h. Infiltration of individual leaves was carried out on 4-5 weeks old recipient plants using a 2 ml syringe by pressing the tip of the syringe (without a needle) against the abaxial surface of the leaf. Infiltrated plants were maintained at 21-22° C. with a photoperiod of 16 h light and 8 h dark.

### Example 3

### Inoculation of Plants by Wound-and-Agrospray

Agrobacterium harboring pKA20 was prepared as described in Example 2 but the infection medium was supplemented with Tween 20 [0.01% (v/v)]. The suspension of Agrobacterium was sprayed onto plants using an air-brush, Model 200 NH, connected to a compressor that produced 20-50 PSI pressure (Badger Air-Brush Co, Illinois, USA). Plants were wounded by slightly scoring the adaxial surfaces of upper leaves with the edge of a plastic pot label.

### Example 4

### Inoculation of Plants by Agrospray

Agrobacterium harboring pKA20 was prepared as described in Example 2 but the infection medium was supplemented with Tween 20 [0.01% (v/v)]. The suspension of Agrobacterium was sprayed onto plants using an air-brush, Model 200 NH, connected to a compressor that produced 20-50 PSI pressure (Badger Air-Brush Co, Illinois, USA). Plants were not wounded in any way prior to application of the Agrospray.

### Example 5

### RNA Analysis

Total RNA was extracted using Trizol Reagent (Invitrogen Corporation, Carlsbad, Calif., USA). Total RNA (5-10 μg/lane) was separated by 1.2% formaldehyde-agarose gels, and blotted onto Hybond-N nylon membrane (Amersham Biosciences, Piscataway, N.J., USA) by following the manufacturer's protocols. Blots were hybridized with a probe containing the full-length vcp coding sequence labeled with ³²P-[dCTP] using the Prime-It II® random priming kit (Stratagene, Cedar Creek, Tex., USA), and washed according to the manufacturer's instruction.

### Example 6

### Isolation and Detection of siRNAs

Short interfering RNAs (siRNA) were detected as described by Dalmay et al. (2000) with modifications as described below. For northern blot analysis, 10 μg of low molecular weight RNA was mixed with loading dye containing formamide and bromophenol blue (final concentration 0.025%), and subjected to electrophoresis in a 15% polyacrylamide/7 M urea Ready Gel (Bio-Rad, Hercules, Calif., USA) in 0.5× TBE buffer at 180 V. The RNA was electroblotted onto a Hybond XL nylon membrane at 100 V for 1 h in 0.5× TBE. Single-stranded ³²P-UTP labeled RNA probes were made from the vcp gene cloned in a pBSSKII vector using Promega (Madison, Wis., USA) Riboprobe system according to the manufacturer's instructions. Antisense vcp RNA was transcribed in vitro using T3 polymerase. The probes were hydrolyzed, and hybridization was carried out as described (Dalmay et al., 2000).

### Example 7

### Immuno-Blot Analysis

Total soluble protein was extracted, using a buffer modified from that of Biemelt et al. (2003). Total soluble protein was quantified using the Protein Assay Dye Reagent Concentrate® (Bio-Rad). Samples from infiltrated and systemically infected leaves were collected separately for analyses involving comparison of L1 accumulation in these tissues from a single plant. In other experiments, in which the goal was to measure the recombinant protein within the whole plant, quantitative L1 assays were carried out on extracts from leaf samples from a single whole plant. Crude protein extracts (11-25 μg per lane) were separated on 10% Tris-HCl gels and transferred to 0.45 μM nitrocellulose membranes. Bound L1 protein was detected by incubating the blots with a primary antibody “B1,” which is a monoclonal antibody raised against an epitope present on the linear L1 protein, followed by incubation with a secondary antibody and chemiluminescence reagents from the WesternBreeze™ kit (Invitrogen), according to the manufacturer's protocol.

### Example 8

### Chloroplast Isolation and Detection of L1 Protein

Chloroplasts were isolated from infiltrated leaves essentially as described (Mills and Joy, 1980). TEV-B and Xanthi tobacco plants were infiltrated with Agrobacterium containing the pKA20 construct. Twelve days after infiltration, the infiltrated and control plants were maintained in the dark for 48-60 h to reduce chloroplast starch levels. Approximately 4 grams of leaf tissue comprising a bulk sample of infiltrated and systemically infected leaves was harvested, washed with ice-cold sterile water, and sliced into small pieces (2 cm×2 cm). The sliced leaf pieces were placed in 50 ml ice-cold chloroplast isolation buffer (CIB) (330 mM sorbitol, 50 mM Tris-HCl, 1 mM MgCl₂, 2 mM EDTA, 0.1% BSA, and 25 mM β-mercaptoethanol, pH 8.0) and were homogenized using a small Warring commercial Blender (Fisher, Pittsburgh, Pa., USA) with two bursts of three seconds each, at the low speed setting. The homogenate was filtered through three layers of cheesecloth and three layers of Miracloth (Calbiochem, La Jolla, Calif., USA). The filtrate was aliquoted into 50 ml Falcon centrifuge tubes and centrifuged at 1500 g for 7 min at 4° C. The supernatant was discarded and the pellets were re-suspended in 1.5 ml CIB per tube. The suspension was carefully layered on 40-80% discontinuous Percoll (MP Biomedicals LLC, Aurora, Ohio, USA) gradients (5 ml and 4 ml respectively) and centrifuged at 2000 g for 7 min at 4° C. Intact chloroplasts were collected at the interface of 40% and 80% Percoll layers and suspended with 1 ml CIB without BSA and β-mercaptoethanol, and centrifuged at 2500 g for 6 min at 4° C. The supernatant was discarded and three 2.5 mm glass beads (Biospec, Bartlesville, Okla., USA) were added to the pellet in 1.5 ml Eppendorf tube, which was then flash frozen in liquid nitrogen. The pellet was thawed on ice and homogenized in a Silamat S5 amalgamator (Ivoclar Vivadent, Amherst, N.Y., USA). Finally, the pellet containing the chloroplast fraction was resuspended in a modified protein extraction buffer (Biemelt et al., 2003). Eleven micrograms of total soluble protein was separated on a 10% Tris-HCL gel, transferred to nitrocellulose, and immuno-blot analysis was carried out as described above.

### Example 9

### Protein Extraction and Immunoblot Analysis

Tissues were harvested at 12 d.p.i. and protein was isolated for Western analysis as briefly described below. At 12 d.p.i., tissues were isolated and weighted. Following weighing, the tissues were ground in a mortar and pestle in the presence of liquid nitrogen. After grinding, about two volumes of protein extraction buffer based upon sample weight were added to the ground plant. For example, if the isolated tissue sample weighted 10 grams, then 20 ml of extraction buffer would be added to the ground plant material. The protein extraction buffer was well mixed with the ground plant material, and then the sample mixture was vortexed briefly. Following vortexing, the sample mixture was mixed slowly at 4° C. for 10 minutes. The sample mixture was then centrifuged at 10,000×g for 10 minutes at 4° C. The supernatant, comprising the protein extact, was then carefully decanted. The protein concentration in the supernatant was determined using a form of the Bradford protein assay (Bio-Rad Protein Assay Dye Reagent Concentrate®, Bio Rad Laboratories, Inc.).

Several types and compositions of the protein extraction buffer may be used in this procedure. Typically, for the experiments described in the examples herein, the protein extraction buffer comprised the following: 50 mM Tris·HCl, pH 7.0, 1 mM EDTA pH 8.0, 1 mM EGTA, 5 mM MgCl, 15% (v/v) glycerol, 14 mM β-mercaptoethanol, and a protease inhibitor cocktail (Sigma-Aldrich, Inc.) comprising a mixture of protease inhibitors with broad specificity for the inhibition of serine, cysteine, aspartic, and metalloproteases, and aminopeptidases. The protease inhibitor cocktail contained 4-(2-aminoethyl)benzenesulfonyl fluoride (AEBSF), bestatin, pepstatinA, E-64, leupeptin, and 1,10-phenanthroline.

Immunoblot analysis (also called “Western blotting”) of protein extracts was carried out on protein samples separated by denaturing gel electrophoresis, followed by transfer (or “blotting”) of the separated proteins to a solid matrix, such as nitrocellulose. Typically, a suitable protein sample, for example, a protein extracts, is mixed with a sample buffer to yield a mixture comprising 62.5 mM Tris (pH 6.8), 2% (w/v) SDS, 5% (v/v) β-mercaptoethanol, 10% (v/v) glycerol, and 0.25% (w/v) bromophenol blue, and then the mixture is heated at 100° C. for 10 minutes. The denatured protein sample is then separated using sodium dodecylsulfate (“SDS”) polyacrylamide gel electrophoresis (“SDS-PAGE”). The resolving gel comprises a solution prepared by mixing 4.0 ml of distilled water, 2.5 ml 1.5M Tris·HCl (pH 8.8), 100 μl 10% (w/v) SDS, 3.3 ml 30% (w/v) acrylamide/bis-acrylamide (29:1 acrylamide: bis-acrylamide), 65 μl 10% (w/v) ammonium persulfate (“APS”), and 5 μl N,N,N′,N′-Tetramethyl-1-,2-diaminomethane (“TEMED”). The foregoing solution is sufficient for the preparation of two resolving gels (Mini-PROTEAN® gel electrophoresis apparatus, Bio-Rad Laboratories, Inc., Hercules, Calif.). The stacking gel comprises a solution prepared by mixing 2.98 ml distilled water, 1.26 ml 0.5M Tris·HCl (pH 6.8), 50 μl 10% (w/v) SDS, 670 μl 30% (w/v) acrylamide/bis-acrylamide (29:1 acrylamide: bis-acrylamide), 35 μl 10% (w/v) APS, and 5 μl TEMED. The electrophoresis buffer comprised 25 mM Tris, 192 mM glycine, and 3.5 mM SDS. Electrophoresis was carried out at 125 VDC at constant voltage. Following gel electrophoresis, the separated proteins are transferred onto nitrocellulose (Trans-Blot Transfer Medium®, 0.45 Bio-Rad Laboratories, Inc.) at 100 VDC at constant voltage using a Mini Trans-Blot® transfer unit (Bio-Rad Laboratories, Inc.). The transfer buffer used comprised 25 mM Tris, 192 mM glycine, and 20% (v/v) methanol. After transfer, the nitrocellulose blot was stained with Ponceau S (Sigma-Aldrich, Inc.; CAS No. 6226-79-5; molecular formula C₂₂H₁₂N₄Na₄O₁₃S₄) in order to assess the extent of transfer. Detection of specific protein bands is carried out using first a primary antibody (e.g., B1 antibody raised against an epitope present on the linear L1 protein), is an antibody specific for an epitope on a specific protein that may be in the protein sample. A secondary antibody is used that recognizes the first antibody and is conjugated to an enzyme (e.g. goat anti-mouse antibody conjugated to the enzyme horseradish peroxidase), followed by using of a chemiluminescence reagent that emits light in the presence of a suitable enzyme.

In some experiments, processing of the immunoblot and detection of protein bands was carried out using buffers, secondary antibody, and chemiluminescence reagents found in the WesternBreeze™ Kit, following the manufacturer's protocol (Invitrogen Corporation, Carlsbad, Calif.). The blot was then exposed to film (Kodak Biomax XAR Film®, Kodak Corporation, Rochester, N.Y.) for 1, 2, 3, 4, 5, 6, 7, 8, 9, 10, 30, 60, 120, 300, or 600 seconds to visualize antibody-bound protein bands. The length of exposure was dependent upon the signal strength of the detected proteins.

Alternatively, in other experiments, nitrocellulose blot was blocked using a solution comprising 5% (w/v) skim milk in TBS (20 mM Tris, 500 mM NaCl, pH 7.4) or PBS (10 mM sodium phosphate, 150 mM NaCl, pH 7.4). Following washing, the nitrocellulose blot was incubated with a solution comprising the primary antibody diluted appropriately in 5% (w/v) skim milk in TBS or PBS. The nitrocellulose blot is washed 3 times for 10 minutes per wash with TBS-T (TBS containing 0.05% (w/v) Tween-20, wherein Tween-20 comprises poly(oxyethylene)(20)-sorbitane monooleate, C₅₈H₁₁₄O₂₆, CAS No. 9005-64-5), and then incubated with secondary antibody, for example, goat anti-mouse antibody linked to alkaline phosphatase. The nitrocellulose blot was then washed in TBS-T. Detection of protein bands was carried out using chemiluminescence using the Pierce SuperSignal West Femto Maximum-Sensitivity Substrate (Pierce Chemical Company, Rockland, Ill.). The blot was then exposed to film (Kodak Biomax XAR Film®, Kodak Corporation, Rochester, N.Y.) for 1, 2, 3, 4, 5, 6, 7, 8, 9, 10, 30, 60, 120, 300, or 600 seconds to visualize antibody-bound protein bands. The length of exposure was dependent upon the signal strength of the detected proteins.

### Example 10

### Agrobacterium-Mediated Transient Gene Expression

N. tabacum cv Xanthi and TEV-B plants were infiltrated with Agrobacterium as described with the constructs described in Example 1. TEV-B plants infiltrated with any of the constructs showed systemic PVX symptoms upon visual inspection 7 days after inoculation. In contrast, N. tabacum cv Xanthi plants infiltrated with any of the constructs did not show any systemic PVX symptoms at 12 d.p.i.

### Example 11

### Immunoblot Analysis of Protein Expression in Infiltrated and Systemic Leaves from Tobacco cv. Xanthi and TEV-B Plants

Western blot analysis was carried out on the same plants described in Example 9. Tissues were harvested at 12 d.p.i. and protein was isolated for Western analysis. Gel electrophoresis was carried out using SDS-PAGE on a 7.5% acrylamide gel after loading 50 μg of total protein per lane. After transfer to a membrane, the blot was probed with a monoclonal antibody (B1) to an epitope present in the primary (linear) L1 peptide. The results of Western analysis show that protein expression was detected in both infiltrated and systemic tissues collected from the TEV-B plants infiltrated with the expression construct containing a chloroplast-targeting protein. N. tabacum cv Xanthi plants infiltrated with the same expression construct also show expression of the L1 protein, but only in the infiltrated leaf tissue. There was no detection of L1 protein under these conditions in any other than infected leaves of N. tabacum cv Xanthi plants infiltrated with this construct.

### Example 12

### Northern and Western Analysis of Infiltrated and Systemic Leaves from Tobacco cv. KY14 Plants

A commercial variety of tobacco, N. tabacum cv. KY14 was infiltrated with the constructs described in Example 1. Northern analysis of total RNA isolated from tissues collected at 12 d.p.i. indicate revealed vcp-containing mRNA transcript only in extracts from infiltrated leaves. No mRNA was detected in the systemic tissues. The tissues that were positive for the northern analysis were also analyzed by western blot. In this genotype, only the pKA20 construct produced detectable L1 protein.

### Example 13

### Subcellular Targeting of L1 Recombinant Protein

The L1 protein was targeted for retention in the endoplasmic reticulum (ER). In these constructs, a 5′ un-translated region of tobacco etch virus (TEV leader sequence) and the signal sequence (SP) from the tobacco pathogenesis related protein PR1a from pGA748 (Xu et al., 2002), was fused in-frame with the vcp gene. Moreover, the endoplasmic reticulum retrieval signal KDEL coding sequence was included in the PCR reverse primer so as to be fused at the 3′ terminus of the vcp gene. The resulting fusion sequence (TEV::SP::vcp::KDEL) was cloned in pCR2.1 vector and was verified by sequence analysis. The fusion sequence was released by cutting pCR2.1 vector with EcoRI, blunted, and inserted at the blunted NotI site in vector pGR106. For apoplast targeting, a similar approach was used to make fusion gene construct as above (ER targeting) but without KDEL at the 3′ of the vcp gene. The fusion gene construct was sequence verified and cloned in pGR106 at the blunted NotI site.

### Example 14

### Tobacco Chloroplast Transit Peptide Sequence

The chloroplast transit peptide (CTP) coding sequence for the ribulose-1,5-bisphosphate carboxylase (rubisco) small subunit of tobacco was isolated by PCR amplification from genomic DNA of N. tabacum cv. KY14 using upstream primer containing an XhoI site 5′CTCGAGATGGCTTCCTCAGTTCTTTCCTCT3′ (SEQ ID NO.8) and downstream primer 5′GCATTGCACTCTTCCGCCGTTGCTGG 3′ (SEQ ID NO.9). The PCR amplified CTP coding fragment (171 bp) was cloned into pCR2.1 vector as described by the manufacturer (Invitrogen, Carlsbad, Calif.) and the authenticity of the sequence was verified by sequence analysis. PCR based precise in-frame fusion of CTP and a 550 by fragment of vcp was carried out using three primers [P1: 5′CTCGAGATGGCTTCCTCAGTTCTTTCCTCTG3′ (SEQ ID NO.10), P2: 5′AGCAGGAAGCCAAACAGCCATGCATTGCACTCTTCCGCCG3′ (SEQ ID NO.11), and P3: 5′ACCGGTAACAGAAATACCAAGTGGTTGACCT 3′ (SEQ ID NO.12) as described (Yon and Fried, 1989). The P1 primer was a forward primer of CTP and the P2 primer was to make a fusion between the 3′ of CTP and 5′ of vcp coding region. The P3 primer was a complementary sequence of the vcp coding sequence which is downstream of a unique AgeI restriction site convenient for the construction. The PCR product (550 bp) was cloned into pCR2.1 vector (Invitrogen) and verified by sequence analysis. The resulting plasmid carrying the fusion fragment and the pBSK-vcp were both digested with AgeI and EcoRV enzymes. The original sequence of vcp between EcoRV and AgeI in pBSK-vcp was replaced with the fusion fragment. The resulting fusion gene (CTP::vcp, 1.7 kb) was eventually inserted at the blunted NotI site of pGR106.

### Example 15

### Chloroplast Transit Peptide Sequences

Targeting of recombinant proteins, including, but not limited to the vcp gene product and any of the other recombinant proteins in accordance with the present invention, to the chloroplast may be accomplished with other CTP sequences. Example 13 describes the use of the tobacco CTP sequence for targeting the vcp gene product to the chloroplast. Other CTP sequences suitable for chloroplast target include those listed in Table 1, which displays the nucleotide sequences encoding CTP sequences from pea (Genbank Accession No. X008060), lettuce ((Genbank Accession No. D14001), and rice (X07515), in addition to tobacco (Genbank Accession No. X02353). In addition to the native CTP sequences listed in Table 1, it is also possible, in accordance with the present invention, to utilize synthetic, chimeric, variant and homologous CTP sequences. In accordance with the present invention, suitable CTP sequences include homologous CTP sequences derived by consensus alignment of two or more known CTP sequences. For example, CTP sequences from multiple species were subjected to multiple sequence alignment as shown in Table 2, which was then utilized to obtain consensus sequences of varying degrees of homology based upon percentage threshold values. The consensus sequences so derived are suitable, in accordance with the present invention, for use in targeting recombinant proteins to chloroplasts.

TABLE 1
Alignment of DNA Sequences Encoding a Chloroplast
Transit Peptide from Various Species*

| | | |
|---|---|---|
| Pea¹ | ATGGCTTCTATGATATCCTCTTCCGCTGTG | 60 |
| | ACAACAGTCAGCCGTGCCTCTAGGGGGCAA | |
| Lettuce² | ATGGCCTCCATC---TCCTCCTCAGCCATC | 57 |
| | GCCACCGTCAACCGGACCACCTCCACCCAA | |
| Tobacco³ | ATGGCTTCCTCA---GTTCTTTCCTCTGCA | 57 |
| | GCAGTTGCCACCCGCAGCAATGTTGCTCAA | |
| Rice⁴ | ------------------------------ | 27 |
| | ---ATGGCCCCCTCCGTGATGGCGTCGTCG | |
| | * * * | |
| Pea | TCCGCCGCAGTGGCTCCATTCGGCGGCCTC | 117 |
| | AAATCCATGACTGGATTCCCAGTGA---AG | |
| Lettuce | GCTAGCTTGGCAGCTCCATTCACCGGCCTC | 117 |
| | AAGTCTAACGTAGCTTTCCCAGTTACCAAG | |
| Tobacco | GCTAACATGGTTGCACCTTTCACTGGCCTT | 117 |
| | AAGTCAGCTGCCTCATTCCCTGTTTCAAGG | |
| Rice | GCCACCACCGTCGCTCCCTTC-CAGGGCTC | 85 |
| | * * * ** ** *** ** ** | |
| | AAGTCCACCGCCGGCATGCC-GTCGCCCGC | |
| | ** ** * ** ** | |
| Pea | AAGGTCAACACTGACATTACTTCCATT | 171 |
| | ACAAGCAATGGTGGAAGAGTAAAGTGC | |
| Lettuce | AAGGCTAACAATGACTTTTCATCCCTA | 171 |
| | CCCAGCAACGGTGGAAGAGTACAATGC | |
| Tobacco | AAGCAAAACCTTGACATCACTTCCATT | 171 |
| | GCCAGCAACGGCGGAAGAGTGCAATGC | |
| Rice | CGTCCGAACTCCAGCTTCGG---CAAC | 135 |
| | *** * * * | |
| | GTCAGCA-TGGCGGCAGGATCAGGTGC | |
| | **** ** ** ** * *** | |

*Sequence alignment was carried using CLUSTAL W (v1.82) using sequence of the chloroplast transit peptide of the small subunit of ribulose-1,5-bisphosphate carboxylase (RuBisCO) from the indicated plant species; an asterisk below the aligned sequences indicates a position in the aligned sequence wherein the nucleotide at the indicated position is conserved in all four of the aligned sequences.
¹SEQ ID NO. 13
²SEQ ID NO. 14
³SEQ ID NO. 1
⁴SEQ ID NO. 15

TABLE 2
Alignment of Chloroplast Transit Peptides
from Various Species*

| | |
|---|---|
| Pea_cTP¹ | -MASMISSSAVTTVSRASTVQSAAVAPFGG |
| | LKSMTGFPV-KKVNTDITSITSNGGRVKCM |
| Lettue² | -MAS-ISSSAIATVNRTTSTQASLAAPFTG |
| | LKSNVAFPVTKKANNDFSSLPSNGGRVQCM |
| Tobacco_cTP³ | MASSVLSSAAVATRSNV--AQANMVAPFTG |
| | LKSAASFPVSRKQNLDITSIASNGGRVQCM |
| Arabidopsis_cTP⁴ | MASSMLSSTAVVTSP----AQATMVAPFTG |
| | LKSSASFPVTRKANNDITSITSNGGRVSCM |
| Spinach_cTP⁵ | MASSVLSSAAVATVSRTP-AQASMVAPFTG |
| | LKSTVGFPATKK-NDDITSLASNGGRVQCM |
| Wheat_cTP⁶ | MAPAVMASS------------ATTVAPFQG |
| | LKSTAGLPVSRRSRGS-LGSVSNGGRIRCM |
| Rice_cTP⁷ | MAPTVMASS------------ATSVAPFQG |
| | LKSTAGLPVSRRSTNSGFGNVSNGGRIKCM |
| consensus/90% | .hss.huSs............us.sAPFtG |
| | LKS.suhPs.++.p.s..u.sSNGGRlpCM |
| consensus/80% | .hsohhuSu............AshVAPFpG |
| | LKSssuhPVo++.ssshhu.sSNGGRlpCM |
| consensus/70% | MAuSh1SSoA1sT.s....sQAohVAPFsG |
| | LKSssuFPVo+KsNsDhoSlsSNGGRVpCM |
| consensus/60% | MAuSh1SSoA1sT.s....sQAohVAPFsG |
| | LKSssuFPVo+KsNsDhoSlsSNGGRVpCM |
| consensus/50% | MAuSV1SSSAVsTssps..sQAohVAPFTG |
| | LKSoAGFPVoRKuNsDITSlsSNGGRVIDCM |

*Sequence alignment was carried using CLUSTAL W (v1.82) using sequence of the chloroplast transit peptide of the small subunit of ribulose-1,5-bisphosphate carboxylase (RuBisCO) from the indicated plant species. The consensus sequence for the indicated threshold levels of homology was calculated using the Consensus algorithm available from EMBL, Universitat Heidelberg.
¹SEQ ID NO. 16
²SEQ ID NO. 17
³SEQ ID NO. 18
⁴SEQ ID NO. 19
⁵SEQ ID NO. 20
⁶SEQ ID NO. 21
⁷SEQ ID NO. 22

### Example 16

### Lysis and Extraction of Isolated Intact Chloroplasts

Intact chloroplasts were isolated by the method described, in Example 8. In this experiment, TEV-B plants were infiltrated with the construct described in Example 1 and compared to control plants which were not infiltrated. Following isolation of intact chloroplasts as described, supra, the intact chloroplasts were frozen in liquid nitrogen with 3-4 glass beads (2.5 mm diameter, BIOSPEC Products, Inc., Bartlesville, Okla.). The frozen intact chloroplasts were then thawed, 100-200 μL of protein extraction buffer was added, and the suspension was homogenized in a Silamat S5 amalgamator (Ivoclar Vivadent, Amherst, N.Y.). The homogenate was then centrifuged at 9,000×G for 30 mins at 4° C. The supernatant was collected and protein concentration was quantified using a Bio-Rad Protein Assay (Bio-Rad, CA). Samples were loaded onto the gel, containing approximately 10-25 μg total protein per lane. Following SDS-PAGE, the gels were then used for immunoblot (Western) analysis, using B1 monoclonal antibody specific to an epitope on the linear (primary) structure of the L1 molecule. The results show enrichment of the recombinant protein in the extracts from intact chloroplasts. Further, the results show the CTP sequence of the full-length CTP::vcp protein appears to have been correctly cleaved from the protein accumulated in the chloroplasts.

### Example 17

### Agroinfiltration with Agrobacterium

Agrobacterium strain GV3101 containing gene constructs as shown in FIG. 1 was grown and prepared for agroinfiltration as described in Example 2. The Infection Medium described in Example 2 was supplemented with Tween 20 [0.01% (v/v)]. The suspension of Agrobacterium was sprayed onto homozygous TEV-B plants using a hand sprayer or air-brush (Badger Air-Brush Co, IL), with or without damaging the leaf, with or without slightly pressing the leaves using a 1 ml syringe without a cannula. Leaf samples were collected and RNA analysis was carried out as described, supra. The results show bulk infiltration of plant leaves using only light mechanical pressure followed by spraying with a suspension of Agrobacterium.

### Example 18

### vcp Gene with Codons Optimized for Expression in Plant Cells

The L1 gene from canine oral papillomavirus (COPV), which encodes the primary coat protein L1, was modified to increase translational efficiency in tobacco plants by the replacement of codons associated with rare tRNAs in tobacco with codons associated with higher abundance tRNAs in tobacco. The replacement of codons in the L1 gene associated with rare tRNAs resulted in the replacement of approximately 21% of the native codons. The optimized L1 gene construct was assembled by Aptagen, Inc. The mutated L1 gene, optimized for expression of protein in tobacco, is designated “vcp.” Although the nucleic acid sequence was changed to alter codon usage, the amino acid sequence encoded by the vcp gene is identical to the amino acid sequence of the wild-type COPV L1 protein. A sequence alignment of the vcp gene with the native COPV L1 gene is shown in Table 3. The alignment in Table 3 was carried out using BESTFIT software as implemented in the GCG/Wisconsin Package. BestFit uses of the local homology algorithm of Smith and Waterman (Advances in Applied Mathematics 2; 482-489 (1981)) to find the best segment of similarity between two sequences.

TABLE 3
Sequence Alignment of the COPV L1 gene and vcp*

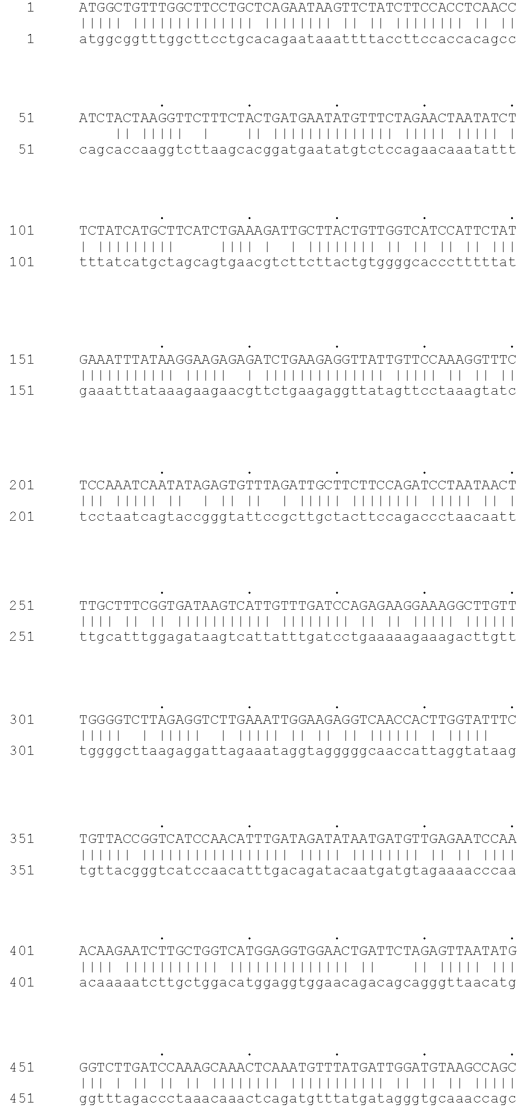

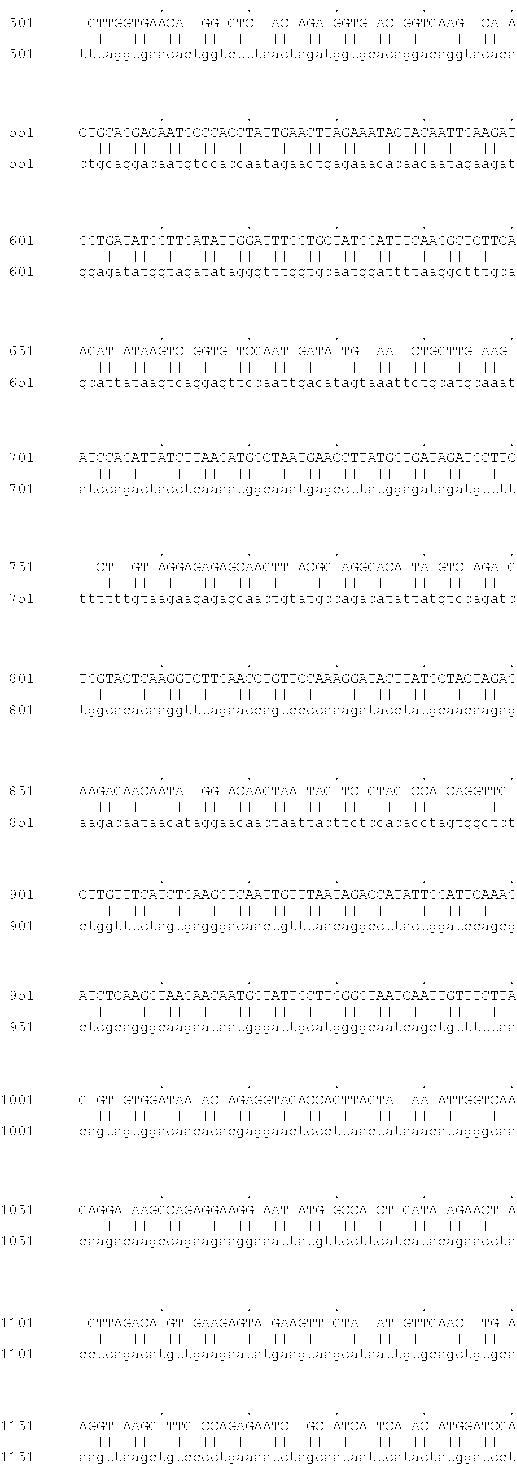

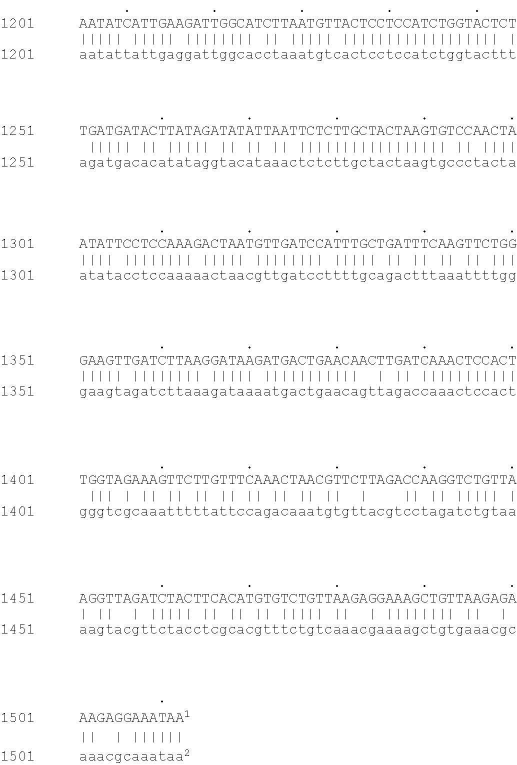

*The top sequence in the alignment is that of vcp and the lower sequence in the alignment is that of the COPY L1 gene.
¹SEQ ID NO. 3
²SEQ ID NO. 23

Discussed below are results of the experiments of Examples 1-18:

In the present invention, the amplicon-plus system has been modified in several important ways that permit expression and accumulation of significant amounts of a labile recombinant protein in as little as 1-2 weeks. First, a replicating amplicon vector carrying the gene of interest, vcp (a synthetic version of the COPV L1 gene), was introduced into tobacco plants (transgenic line TEV-B) by infiltrating leaves with a suspension of Agrobacterium carrying the amplicon, instead of delivering the amplicon by stable transformation. The system was also modified to target L1 to various cellular compartments by creating fusions between the protein of interest and different transit peptides. With these modifications, L1 protein accumulated in the plants most successfully when the protein was targeted to the chloroplasts.

Existing agroinfiltration systems were inefficient and unlikely to be economically viable (Pogue et al., 2002). In order to address this shortcoming, and to improve the utility and economic value of the “amplicon-plus targeting technology” (APTT) system for large-scale production applications, a high-throughput “wound and agrospray” method was developed for Agrobacterium inoculation of tobacco plants, and an “agrospray only” method was developed to facilitate large-scale inoculation of the alternative host N. benthamiana.

L1, the primary coat protein from canine oral papillomavirus (COPV) was chosen as the model protein with which to develop and test APTT for several reasons. First, effective vaccines against papillomaviruses can be made using the L1 coat protein of the target virus, and COPV has been shown to be an excellent model for this type of vaccine (Suzich et al., 1995). Importantly, recombinant L1 is broadly susceptible to factors, such as enzymatic proteolysis, that result in its degradation, thus reducing recovery of the recombinant protein from various sources (Biemelt et al., 2003; Warzecha et al., 2003). L1 therefore serves as a useful model with which to test the efficacy of targeting the protein to various sub-cellular locations as a means of increasing yield of recombinant product from plants.

### Agroinfiltration of TEV-B Plants to Express COPV L1 Protein

Agrobacterium infiltration was used to infect TEV-B plants with various PVX amplicon constructs carrying the vcp gene (FIG. 1).

TEV-B, a tobacco line derived from N. tabacum cv. ‘Xanthi’ that has been stably transformed with a modified form of the P1/HC-Pro gene from tobacco etch virus, was chosen for agroinfiltration experiments because PTGS is effectively eliminated, without the deleterious phenotypic changes associated with use of the un-modified form of P1/HC-Pro (Mallory et al., 2002). Both targeted and non-targeted constructs were tested in an attempt to maximize L1 accumulation. Targeted amplicon gene fusion constructs (FIG. 1) included targeting sequences that directed the L1 protein to the chloroplast, endoplasmic reticulum (ER), or apoplast. Non-transgenic tobacco (control) plants were also infiltrated with Agrobacterium strains containing these constructs.

All infiltrated TEV-B plants showed symptoms similar to those of virus infection on both infiltrated leaves and other leaves of the plants, indicating “systemic infection”. Symptoms were observed on both infiltrated and systemically infected leaves 7-12 days post infiltration (d.p.i.). Symptoms similar to those of systemic infection were only observed at the site of infiltration on the leaves of non-transgenic control plants of tobacco cv. ‘Xanthi’. RNA analysis revealed the presence of large amounts of viral RNAs with homology to vcp in the infiltrated leaves of both TEV-B and Xanthi plants when they were infiltrated with each of the constructs.

Similar levels of RNA were detected in systemically infected leaves of the TEV-B plants but not in control Xanthi plants at 12 d.p.i. By 18 d.p.i. RNA levels had dropped significantly, with the highest levels observed in plants infected with pKA20, which incorporated a coding sequence expressing a fusion of L1 with a chloroplast targeting protein.

Immuno-blot analysis revealed significant accumulation of chloroplast-targeted L1 protein in both infiltrated leaves and systemically infected leaves from TEV-B plants, but L1 accumulated only in the infiltrated leaves of the control Xanthi plants. Only very small quantities of L1 protein accumulated in the infiltrated leaves of TEV-B or Xanthi plants infiltrated with Agrobacterium carrying constructs other than pKA20.

The level of L1 protein in un-inoculated control Xanthi plants, and in Xanthi, TEV-B and N. benthamiana plants infiltrated with pKA20 was measured by image analysis of immuno-blots of extracts from whole plants. The level of L1 protein in TEV-B plants was estimated to be about 3 ng/μg total soluble protein or 0.3% TSP while L1 was barely detectable in the non-transgenic Xanthi plants. In N. benthamiana, the level of protein was estimated to be 0.04% TSP.

The L1 protein accumulated preferentially in the chloroplasts when pKA20 was used to infiltrate TEV-B plants. Results of an immuno-blot comparing L1 levels in total leaf protein with that in protein isolated from leaf extracts in which chloroplast content had been enriched indicated that L1 accumulated preferentially in the chloroplasts. The chloroplast-targeted L1 protein and an authentic L1 standard exhibited essentially identical electrophoretic mobility, indicating that the transit peptide was correctly cleaved after the fusion protein entered the chloroplasts.

The lack of L1 protein accumulation in Xanthi plants was due to PTGS of the viral amplicon RNA in these plants. RNA blot analysis of low molecular weight RNA showed that PTGS-associated siRNAs corresponding to the PVX vector did accumulate in the infiltrated leaves of Xanthi plants, but not in TEV-B plants.

The time course of systemic infection was investigated in the TEV-B plants following Agrobacterium infiltration of the two bottom leaves of the plants. Northern blot analysis detected the presence of viral RNA in the infiltrated leaves 3 d.p.i. There was no substantial difference in the amount of viral RNA accumulated over the period between 4 d.p.i. and 12 d.p.i. in these leaves. In systemically infected leaves, the viral RNA was first detected 6 d.p.i., and the greatest accumulation was observed by 12 d.p.i.

Expression of COPV L1 Protein with PVX Amplicon in Nicotiana benthamiana

PVX amplicon RNA replication and movement, and L1 expression were evaluated in model plant N. benthamiana to compare function of the APTT system in this species with that in tobacco. Four-week-old, un-transformed N. benthamiana plants were infiltrated with Agrobacterium carrying each of the various constructs shown in FIG. 1. In contrast to N. tabacum cv. Xanthi plants infiltrated with these vectors, symptoms of systemic infection were observed in all infiltrated N. benthamiana plants by 7 d.p.i. Viral RNAs that hybridized with a vcp probe were detected in both infiltrated and systemically infected leaves. However, significant degradation of the viral RNA was observed in northern blots of extracts from systemically infected leaves, while most of the viral RNA appeared to be intact in the infiltrated leaves.

Interestingly, even though the viral RNA appeared to be degraded in the systemically infected leaves, L1 protein accumulated in both infiltrated and systemically infected leaves from plants infiltrated with the chloroplast-targeting construct, pKA20. Infiltration with other constructs (FIG. 1) produced no detectable L1 protein. This result was similar to the L1 protein expression data obtained from TEV-B plants infiltrated with this construct, although less L1 protein accumulated in N. benthamiana than in TEV-B plants. Northern blot analysis revealed that systemic movement occurred in N. benthamiana, despite the presence of siRNAs, which accumulated at high levels in plants infected with each construct.

High throughput Inoculation of Tobacco and N. benthamiana by “Agrospray”

To apply APTT to a large-scale commercial production, a simple and efficient method was developed with which it was possible to inoculate a large number of tobacco plants simultaneously. Leaves of the recipient plants were wounded by scratching them lightly with the edge of a plastic pot label, and were then sprayed with an Agrobacterium solution using an airbrush (wound and agrospray). Alternatively, leaves were sprayed without preliminary wounding (agrospray only). Without the use of any mechanized apparatus, these procedures allowed inoculation of plants at a rate of approximately 100 plants/min/person, which is much faster than could be achieved by infiltrating individual plants using a syringe. Symptoms of systemic infection were observed at 12 d.p.i. in TEV-B plants infected with pKA20 using this method (agrospray), but not in Xanthi control plants. Northern blot analysis revealed that TEV-B plants infected using agrospray accumulated viral RNAs with homology to vcp at levels comparable to that observed in TEV-B plants infiltrated using a syringe, but infection only occurred when the plants were wounded prior to infiltration. Imuno-blot analysis showed that recombinant COPV L1 protein accumulated in these plants at levels similar to those observed in plants infiltrated individually with the same Agrobacterium strain using the syringe method.

Similar experiments were carried out with and without mechanical wounding on N. benthamiana plants using Agrobacterium harboring the pKA20 vector. Plants exhibited symptoms of systemic infection at 7 d.p.i. No difference was observed in systemic symptoms regardless of whether or not plants were wounded prior to application of the agrospray suggesting that simply applying an Agrobacterium suspension to leaves as a spray is sufficient to cause infection of N. benthamiana, with subsequent replication and systemic movement of the virus. Northern blot and immuno-blot analyses confirmed that comparable amounts of viral RNAs and L1 protein accumulated in both wounded and unwounded N. benthamiana plants.

Herein is disclosed a novel technology, “Amplicon-plus Targeting Technology” (APTT), for production of high-value recombinant proteins in plants. APTT has two advantages over the original amplicon-plus system (Mallory et al., 2002), from which it was developed. For example, in APTT the self-replicating amplicon may be introduced into recipient plants by infiltration of one or a few leaves on a recipient plant with an Agrobacterium suspension harboring T-DNA encoding the amplicon. Transient expression of this sequence produces self-replicating RNA that can then move systemically to the remainder of the plant. The current invention differs from the original system (Mallory et al., 2002) in which T-DNA encoding the amplicon was stably transformed into the recipient plants. Because stable transformation is avoided, APTT allows production of plants expressing the protein in very little time. Very importantly, infiltration of only a small number of leaves may be sufficient to achieve systemic infection with the amplicon, causing expression of the recombinant protein in essentially all tissues of the plant. This makes it possible to generate a large, reliable supply of uniformly expressing biological material from which the protein of interest can be extracted, a critical requirement for scale-up.

The utility of this system may be further improved by the development of the “agrospray” protocol for inoculation of large numbers of plants. Using this procedure, tobacco plants may be wounded very slightly, and then sprayed with a suspension of Agrobactrium carrying the amplicon of interest. Such a system could readily be mechanized to inoculate very large numbers of plants quickly, to produce a high throughput production system for commercial purposes.

PTGS can severely limit accumulation of recombinant proteins in plants. However, APTT may include the use of a constitutively expressed P1/HC-Pro gene, known to eliminate the accumulation of the viral siRNAs that direct PTGS/VIGS in both tobacco and N. benthamiana (Mallory et al., 2001; Marathe et al., 2000b; Roth et al., 2004). Experimental data indicate that the modified P1/HC-Pro element present in the TEV-B tobacco line effectively reduced PTGS in this system. In tobacco cv. Xanthi, which lacks this suppressor, systemic infection was essentially eliminated. However, systemic infection occurred routinely following infiltration of one or a few leaves of the TEV-B plants.

In the experiments reported above, expression of the recombinant protein occurred quickly following infiltration. A time course experiment revealed peak accumulation of viral RNA and L1 protein in TEV-B plants by 12 days after infiltration, so protein harvest could begin less than two weeks after plants are treated. Furthermore, RNA replication and protein expression persisted to at least 18 days post inoculation, implying that protein recovery could be maximized by allowing additional biomass to accumulate as the plant continues to grow after peak expression levels have been achieved.

The present invention indicated that high levels of recombinant protein expression was achieved quickly in large masses of treated tobacco plants. Although there is a large literature attesting to the utility of tobacco in the production of various recombinant proteins, these data suggest that use of APTT makes tobacco an especially appropriate choice for commercial production of labile, high-value proteins. This is especially true when one also considers that tobacco can quickly produce significant amounts of biomass in a greenhouse.

Another advantage of APTT is that the amplicons can be engineered to incorporate sequences that target the recombinant protein to specific sub-cellular compartments. This may result in a marked increase in recombinant protein yield, presumably by stabilizing the protein and/or protecting it from cellular proteases. Concentration of the protein in specific organelles might also be used to facilitate purification of the recombinant protein.

Poor protein folding, and resulting instability has been shown to reduce accumulation of heterologous proteins in plants (Ma et al., 2003). This problem may be addressed by targeting the proteins to specific organelles (Twyman et al., 2003). The present invention revealed that L1 protein, chosen for these experiments because it is especially labile, accumulated to quite different levels when targeted to different sub-cellular locations, and achieving a maximum when targeted to chloroplasts.

Besides tobacco, other host systems might also be employed in an APTT system. For example, N. benthamiana, which has often been used as a host for plant virus studies because of their susceptibility to infection by RNA viruses, may be employed as a host in an APTT system. Many recombinant proteins have been expressed in this species in recent years (Canizares et al., 2005). Importantly, the inventors disclose that in experiments systemic infection of N. benthamiana with a replicating amplicon could be achieved without the inclusion of an exogenous suppressor of PTGS. Although siRNA was observed, clearly indicating that PTGS was occurring in these plants, this did not prevent replication and systemic movement. Although PTGS appears to have degraded most of the viral RNAs in systemically infected leaves, L1 protein accumulation was observed in systemically infected tissues. This contrasts sharply with observations made in normal Xanthi tobacco plants, where PTGS prevented systemic infection and prevented accumulation of L1 in any but directly infiltrated leaves. Although recombinant protein expression was observed in N. benthamiana without the aid of P1/HC-Pro, systemic infection and overall expression of a recombinant protein in this host could likely be improved by the inclusion of P1/HC-Pro or other suppressor of PTGS (Voinnet et al., 2003).

Another factor that makes N. benthamiana an especially promising alternative host for the APTT system is its readiness with which it is infected by Agrobacterium. The inventors have demonstrated that infection could be achieved by merely spraying the plants with the bacterial suspension. Unlike tobacco, N. benthamiana became infected without preliminary wounding. This feature may further facilitate streamlining the process of generating large, highly-expressing plant populations for commercial protein production.

The inventors showed that heterologous protein expression could be limited by post-transcriptional gene silencing (PTGS) and rapid protein degradation. APTT offers a means for overcoming both of these obstacles in tobacco, by including both the P1/HC-Pro suppressor of PTGS and targeting sequences that permit accumulation of protein in the chloroplast where it is apparently protected from factors that reduce its stability in plant cells.

APTT effectively addresses three of the most important issues required of an effective plant-based transient protein production system. First, suppression of PTGS by P1/HC-Pro prevents accumulation of siRNAs, thus permitting accumulation of large amounts of viral RNA. It also permits systemic movement in tobacco of the self-replicating amplicons carrying the gene of interest. Second, through targeting of the protein to specific sub-cellular locations, recombinant protein, including those known to be highly unstable, can accumulate to high levels, greatly increasing overall efficiency of the system. Third, by delivering a self-replicating amplicon into the plants via the “wound and agrospray” or “agrospray” procedures, it permits production of large amounts of biomass expressing the recombinant protein at minimal cost in a short period of time. This system is ideal not only for routine production of pharmaceutical proteins, but it is also well suited to production of proteins such as vaccines that may be needed quickly and in large amounts in response to health emergencies of natural origin, or those which might result from attack with biological weapons.

Whereas this invention has been described in detail with particular reference to specific embodiments, it will be apparent to those skilled in the art that various modifications and variations can be made in the present invention in light of the above teachings without departing from the scope or spirit of the invention. Other embodiments of the invention will be apparent to those skilled in the art from consideration of the specification and practice of the invention disclosed herein. It is intended that the specification and examples be considered exemplary only, with a true scope and spirit of the invention being indicated by the following claims.

### REFERENCES

Allison, R., R. E. Johnston, R. E. and W. G. Dougherty. The nucleotide sequence of the coding region of tobacco etch virus genomic RNA: evidence for the synthesis of a single polyprotein. Virology. 1986 154, 9-20.

An G., P. R. Ebert, A. Mitra, and S. B. Ha 1988. Binary vectors, p. 1-19. In: S. B. Gelvin and R. A. Schilperoot (eds), Plant Molecular Biology Manual A3. Kluwer Academic Publishers, Dordrecht, The Netherlands.

Anandalakshmi, R., Pruss, G. J., Xin, G., Marathe, R., Mallory, A. C., Smith, T. H., and V. B. Vance. (1998) A viral suppressor of gene silencing in plants. Proc. Natl. Acad. Sci. USA 95:13079-13084.

Angell S. M., and D. C. Baulcombe. (1997) Consistent gene silencing in transgenic plants expressing a replicating potato virus X RNA. EMBO J. 16: 3675-3684.

Barta, A., Sommergruber, K., Thompson, D., Hartmuth, K., Matzke, M. A. and Matzke, A. J. M. (1986) The expression of a nopaline synthase—human growth-hormone chimeric gene in transformed tobacco and sunflower callus-tissue. Plant Mol. Biol. 6, 347-357.

Baulcombe. D. C. (1996). RNA as a target and an initiator of post-transcriptional gene silencing in trangenic plants. Plant Molecular Biology 32, 79-88

Beclin, C., Berthome, R., Palauqui, J.-C., Tepfer, M., and H. Vaucheret. (1998) Infection of tobacco or Arabidopsis plants by CMV counteracts systemic post-transcriptional silencing of non-viral (trans) genes. Virology 252:313-317.

Baulcombe, D. C. (1996) RNA as a target and an initiator of post-transcriptional gene silencing in transgenic plants. Plant Molecular Biology 32:79-88.

Baulcombe, D. C. (2004) RNA silencing in plants. Nature 431:356-363.

Bellucci, M., F. De Marchis, R. Mannucci, and S. Arcioni (2003) Jellyfish green fluorescent protein as a useful reporter for transient expression and stable transformation in Medicago sativa L. Plant Cell Reports 22 (5):328-337.

Ballas, N., S. Broido, H. Soreq H, and A. Loyter. (1989) Efficient functioning of plant promoters and poly(A) sites in Xenopus oocytes. Nucleic Acids Res. 17(19):7891-903.

Brigneti, G., Voinnet, O., Li, W.-X., Ji, L.-H., Ding, S.-W., and D. C. Baulcombe. (1998) Viral pathogenicity determinants are suppressors of transgene silencing in Nicotiana benthamiana. EMBO J. 17:6739-6746.

Biemelt, S., Sonnewald, U., Gaimbacher, P., Willmitzer, L. and Muller, M. (2003) Production of human papillomavirus type 16 virus-like particles in transgenic plants. J. Virol. 77, 9211-9220.

Canizares, M. C., Nicholson, L. and Lomonossoff, G. P. (2005) Use of viral vectors for vaccine production in plants. Immunol. Cell Biol. 83, 263-270.

Christensen, A. H. and P. H. Quail. Sequence analysis and transcriptional regulation by heat shock of polyubiquitin transcripts from maize. Plant Mol Biol 1989 12:619-632.

Christensen, A. H., R. A. Sharrock, and P. H. Quail. (1992) Maize poly-ubiquitin genes: genes, structure, thermal perturbation of expression and transcript splicing, and promoter activity following transfer to protoplast by electroporation. Plant Mol Biol 18:675-689.

Christensen, A. H. and P. H. Quail. (1996) Ubiquitin promoter-based vectors for high-level expression of selectable and/or screenable marker genes in monocotyledonous plants. Transgenic Res. 5(3):213-8.

Dalmay, T., Hamilton, A., Mueller, E. and Baulcombe, D. C. (2000) Potato virus X amplicons in Arabidopsis mediate genetic and epigenetic gene silencing. Plant Cell, 12, 369-379.

Datta, K, N. Baisakh, N. Oliva, L. Torrizo, E. Abrigo, J. Tan, M. Rai, S. Rehana, S. Al-Babili, P. Beyer, I. Potrykus, and S. K. Datta (2003) Bioengineered ‘golden’ indica rice cultivars with beta-carotene metabolism in the endosperm with hygromycin and mannose selection systems. Plant Biotech. J. 1(2):81-90.

Della-Cioppa, G., G. M. Kishore, R. N. Beachy, and R. T. Fraley. (1987) Protein trafficking in plant cells. Plant Physiol. 84:965-968.

Dykxhoorn D. M., C. D. Novina, and P. A. Sharp. (2003) Killing the messenger: Short RNAs that silence gene expression. Nature Reviews Molecular Cell Biology 4:457-467.

Elroy-Stein, O., T. R. Fuerst, and B. Moss. (1989) Cap-independent translation of mRNA conferred by encephalomyocarditis virus 5′ sequence improves the performance of the vaccinia virus/bacteriophage T7 hybrid expression system. Proc Natl Acad Sci USA. 86(16):6126-30.

English, J., G. Davenport, T. Elmayan, H. Vaucheret, and D. Baulcombe. (1997) Requirement of transcription for homology-dependent virus resistance and transinactivation. Plant J. 12:597-603.

Gallie, D. R., C. I. Kado, J. W. B. Hershey, M. A. Wilson, and V. Walbot. (1989) Eukaryotic viral 5′-leader sequences act as translational enhancers in eukaryotes and prokaryotes. In: T. R. Cech (ed.) Molecular Biology of RNA, pp. 237-256. Alan R. Liss, Inc., NY.

Guerineau, F, L. Brooks, and P. Mullineaux. (1991) Effect of deletions in the cauliflower mosaic virus polyadenylation sequence on the choice of the polyadenylation sites in tobacco protoplasts. Mol Gen Genet. 226(1-2):141-4.

Gils, M., Kandzia, R., Marillonnet, S., Klimyuk, V. and Gleba, Y. (2005) High-yield production of authentic human growth hormone using a plant virus-based expression system. Plant Biotechnol. J. 3, 613-620.

Gleba, Y., Klimyuk, V. and Marillonnet, S. (2005) Magnifection—a new platform for expressing recombinant vaccines in plants. Vaccine, 23, 2042-2048.

Grevich, J. J. and Daniell, H. (2005) Chloroplast genetic engineering: Recent advances and future perspectives. Crit. Rev. Plant Sci. 24, 83-107.

Hiatt, A., Cafferkey, R. and Bowdish, K. (1989) Production of antibodies in transgenic plants. Nature, 342, 76-78.

Hoppmann, V., S. Di Fiore, S. Zimmermann, N. Emans, T. Rademacher, R. Fischer, and S. Schillberg. (2002) The potato granule bound starch synthase chloroplast transit peptide directs recombinant proteins to plastids. J. Plant Physiol. 159(10):1061-1067.

Jang, I. C., B. H. Nahm, and J. K. Kim. (1999) Subcellular targeting of green fluorescent protein to plastids in transgenic rice plants provides a high-level expression system. Mol. Breeding 5(5):453-461.

Jang, I. C.; K. H. Lee, B. H. Nahm, and J. K. Kim. (2002) Chloroplast targeting protein of a rice rbcS gene enhances transgene expression. Mol. Breeding 9(2):81-91.

Jobling, S. A. and L. Gehrke. (1987) Enhanced translation of chimaeric messenger RNAs containing a plant viral untranslated leader sequence. Nature 325(6105):622-5.

Joshi, C. P. (1987) Putative polyadenylation signals in nuclear genes of higher plants: a compilation and analysis. Nucleic Acids Res. 15(23):9627-40.

Kasschau, K. D., and J. C. Carrington. (1998) A counter defensive strategy of plant viruses: Suppression of posttranscriptional gene silencing. Cell 95:461-470.

Last, D. I., D. A. Brettell, A. M. Chamberlain, A. M. Chaudhury, P. J. Larkin, E. L. Marsh, W. J. Peacock, and E. S. Dennis. (1991) pEmu: an improved promoter for gene expression in cereal cells. Theor. Appl. Genet. 81, 581-588.

Lommel, S. A., T. L. Kendall T L, Z. Xiong, and R. C. Nutter. (1991) Identification of the maize chlorotic mottle virus capsid protein cistron and characterization of its subgenomic messenger RNA. Virology 181(1):382-5.

Lindbo, J. A., Fitzmaurice, W. P. and della-Cioppa, G. (2001) Virus-mediated reprogramming of gene expression in plants. Curr. Opin. Plant Biol. 4, 181-185.

Lu, R., I. Malcuit, P. Moffett, M. T. Ruiz, J. Peart, A. J. Wu, J. P. Rathjen, A. Bendahmane, L. Day, and D. C. Baulcombe. (2003) High throughput virus-induced gene silencing implicates heat shock protein 90 in plant disease resistance. EMBO J. 22(21):5690-5699.

Macejak, D. G., and P. Sarnow. (1991) Internal initiation of translation mediated by the 5′ leader of a cellular mRNA. Nature. 353(6339):90-4.

Mallory, A. C., L. Ely, T. H. Smith, R. Marathe, R. Anandalakshmi, M. Fagard, H. Vaucheret, G. Pruss, L. Bowman, and V. B. Vance. (2001) HC-Pro suppression of transgene silencing eliminates the small RNAs but not transgene methylation or the mobile signal. The Plant Cell 13:571-583.

Mallory, A. C., G. Parks, M. W. Endres, D. Baulcombe, L. H. Bowman, G. J. Pruss, and V. B. Vance. (2002) The Amplicon-plus system for high-level expression of transgenes in plants. Nature Biotechnology 20:622-625.

Ma, J. K. C., Drake, P. M. W. and Christou, P. (2003) The production of recombinant pharmaceutical proteins in plants. Nat. Rev. Genet. 4, 794-805.

Marathe, R., Anandalakshmi, R., Smith, T. H., Pruss, G. J. and Vance, V. B. (2000a) RNA viruses as inducers, suppressors and targets of post-transcriptional gene silencing. Plant Mol. Biol. 43, 295-306.

Marathe, R., Smith, T. H., Anandalakshmi, R., Bowman, L. H., Fagard, M., Mourrain, P., Vaucheret, H. and Vance, V. B. (2000b) Plant viral suppressors of post-transcriptional silencing do not suppress transcriptional silencing. Plant J. 22, 51-59.

Marillonnet, S., Thoeringer, C., Kandzia, R., R, K. and R., G. (2005) Systemic Agrobacterium tumefaciens-mediated transfection of viral replicons for efficient transient expression in plants. Nat. Biotechnol. 23, 718-723.

Mills, W. R. and Joy, K. W. (1980) Rapid method for isolation of purified, physiologically active chloroplasts, used to study the intracellular-distribution of amino-acids in Pea leaves. Planta, 148, 75-83.

McElroy, D., W. Zhang, J. Cao, and R. Wu. (1990) Isolation of an efficient actin promoter for use in rice transformation. Plant Cell. 2(2):163-71.

McKersie, B. D., Y. R. Chen, M. Debeus, S. R. Bowley, C. Bowler, D. Inze, K. Dhalluin, and J. Botterman. (1993) Superoxide-dismutase enhances tolerance of freezing stress in transgenic alfalfa (Medicago sativa L.). Plant Phyisol. 103(4):1155-1163

McManus M. T. and P. A. Sharp. (2002) Gene silencing in mammals by small interfering RNAs. Nature Reviews Genetics 3:737-747.

Mills, W. R., and K. W. Joy. (1980) A rapid method for isolation of purified physiologically active chloroplasts, used to study the intracellular distribution of amino acids in pea leaves. Planta 148: 75-83.

Mogen, B. D., M. H. MacDonald, R. Graybosch, and A. G. Hunt. (1990) Upstream sequences other than AAUAAA are required for efficient messenger RNA 3′-end formation in plants. Plant Cell. 2(12):1261-72.

Munroe D. and A. Jacobson. Tales of poly(A): a review. Gene 91: 151-158.

Odell, J. T., F. Nagy, and N. H. Chua. (1985) Identification of DNA sequences required for activity of the cauliflower mosaic virus 35S promoter. Nature 313(6005):810-2.

Perlak, F. J., Fuchs, R. L., Dean, D. A., Mcpherson, S. L. and Fischhoff, D. A. (1991) Modification of the coding sequence enhances plant expression of insect control protein genes. Proc. Natl. Acad. Sci. USA, 88, 3324-3328.

Pogue, G. P., Lindbo, J. A., Garger, S. J. and Fitzmaurice, W. P. (2002) Making an ally from an enemy: Plant virology and the new agriculture. Annu Rev. Phytopathol. 40, 45-74.

Proudfoot, N. (1991) Poly(A) signals. Cell. 64(4):671-4.

Roth, B. M., Pruss, G. J. and Vance, V. B. (2004) Plant viral suppressors of RNA silencing. Virus Res., 102, 97-108.

Sambrook, J. and D. W. Russell. (2001) Molecular Cloning: A Laboratory Manual. Cold Spring Harbor Laboratory Press, Cold Spring Harbor, N.Y.

Sablowski, R. W., D. C. Baulcombe, and M. Bevan. (1995) Expression of a flower-specific Myb protein in leaf cells using a viral vector causes ectopic activation of a target promoter. Proc Natl Acad Sci USA. 92(15):6901-5.

Sanfacon, H, P. Brodmann, and T. Hohn. (1991) A dissection of the cauliflower mosaic virus polyadenylation signal. Genes Dev. 5(1):141-9.

Shen, B., R. G. Jensen, and H. J. Bohnert. (1997) Increased resistance to oxidative stress in transgenic plants by targeting mannitol biosynthesis to chloroplasts. Plant Physiol. 113(4):1177-1183.

Suzich, J. A., Ghim, S. J., Palmerhill, F. J., White, W. I., Tamura, J. K., Bell, J. A., Newsome, J. A., Jenson, A. B. and Schlegel, R. (1995) Systemic immunization with Papillomavirus L1 protein completely prevents the development of viral mucosal papillomas. Proc. Natl. Acad. Sci. USA, 92, 11553-11557.

Twyman, R. M., Stoger, E., Schillberg, S., Christou, P. and Fischer, R. (2003) Molecular farming in plants: host systems and expression technology. Trends Biotechnol. 21, 570-578.

Velten, J., L. Velten, R. Hain, and J. Schell. (1984) Isolation of a dual plant promoter fragment from the Ti plasmid of Agrobacterium tumefaciens. EMBO J. 3:2723-2730.

Voinnet, O., Vain, P., Angell, S., and Baulcombe, D. C. (1998) Systemic spread of sequence-specific transgene RNA degradation in plants is initiated by localized introduction of ectopic promoterless DNA. Cell 95:177-187.

Voinnet, O., Pinto, Y. M., and Baulcombe, D. C. (1999) Suppression of gene silencing: A general strategy used by diverse DNA and RNA viruses of plants. Proc. Natl. Acad. Sci. USA 96:14147-14152.

Voinnet, O., Rivas, S., Mestre, P. and Baulcombe, D. (2003) An enhanced transient expression system in plants based on suppression of gene silencing by the p19 protein of tomato bushy stunt virus. Plant J. 33, 949-956.

Warzecha, H., Mason, H. S., Lane, C., Tryggvesson, A., Rybicki, E., Williamson, A. L., Clements, J. D. and Rose, R. C. (2003) Oral immunogenicity of human papillomavirus-like particles expressed in potato. J. Virol. 77, 8702-8711.

Wilcox, C. P., Weissinger, A. K., Long, R. C., Fitzmaurice, L. C., Mirkov, T. E. and Swaisgood, H. E. (1997) Production and purification of an active bovine lysozyme in tobacco (Nicotiana tabacum): Utilization of value-added crop plants traditionally grown under intensive agriculture. J. Agr. Food Chem. 45, 2793-2797.

Xu, H., Montoya, F. U., Wang, Z. P., Lee, J. M., Reeves, R., Linthicum, D. S. and Magnuson, N. S. (2002) Combined use of regulatory elements within the cDNA to increase the production of a soluble mouse single-chain antibody, scFv, from tobacco cell suspension cultures. Protein Expres. Purif. 24, 384-394.

Yon, J. and Fried, M. (1989) Precise gene fusion by PCR. Nucleic Acids Res. 17, 4895-4895.

Zhong, H., F. Teymouri, B. Chapman, S. B. Maqbool, R. Sabzikar, Y. El-Maghraby, B. Dale, and M. B. Sticklen. (2003) The pea (Pisum sativum L.) rbcS transit peptide directs the Alcaligenes eutrophus polyhydroxybutyrate enzymes into the maize (Zea mays L.) chloroplasts. Plant Sci. 165(3):455-462.

1-77. (canceled)

78. A method for producing a polypeptide in a plant, comprising contacting a plant with a composition comprising at least one recombinant amplicon comprising a nucleic acid encoding at least one signal peptide or at least one transit peptide, or both, operably linked to a nucleic acid encoding a polypeptide, wherein the polypeptide is expressed while a suppressor of a post transcriptional gene silencing process is active.

79. The method of claim 78, wherein the composition comprises a recombinant bacterial vehicle transformed with at least one recombinant amplicon.

80. The method of claim 78, wherein the at least one transit peptide is a chloroplast-targeting transit peptide.

81. The method of claim 78, wherein the composition further comprises at least one nucleic acid encoding a suppressor of a post transcriptional gene silencing process.

82. The method of claim 78, wherein the suppressor of a post transcriptional gene silencing process is P1/HC-Pro.

83. The method of claim 78, wherein the plant is transformed with at least one nucleic acid encoding a suppressor of a post transcriptional gene silencing process.

84. A polypeptide produced in accordance with a method comprising contacting a plant with a composition comprising at least one recombinant amplicon comprising a nucleic acid encoding at least one signal peptide or at least one transit peptide, or both, operably linked to a nucleic acid encoding a polypeptide, wherein the polypeptide is expressed while a suppressor of a post transcriptional gene silencing process is active.

85. The polypeptide of claim 84, wherein the composition comprises a recombinant bacterial vehicle transformed with at least one recombinant amplicon.

86. The polypeptide of claim 84, wherein the at least one transit peptide is a chloroplast-targeting transit peptide.

87. The polypeptide of claim 84, wherein the composition further comprises at least one nucleic acid encoding a suppressor of a post transcriptional gene silencing process.

88. The polypeptide of claim 87, wherein the suppressor of a post transcriptional gene silencing process is P1/HC-Pro.

89. The polypeptide of claim 84, wherein the plant is transformed with a nucleic acid encoding a suppressor of a post transcriptional gene silencing process.

90. A plant comprising a recombinant composition, wherein the recombinant composition comprises at least one recombinant amplicon comprising a nucleic acid encoding at least one signal peptide or at least one transit peptide, or both, operably linked to a nucleic acid encoding a polypeptide, wherein the polypeptide is expressed while a suppressor of a post transcriptional gene silencing process is active.

91. The plant of claim 90, wherein the transit peptide is a chloroplast-targeting transit peptide.

92. The plant of claim 90, wherein the recombinant composition further comprises at least one nucleic acid encoding a suppressor of a post transcriptional gene silencing process.

93. The plant of claim 90, wherein the plant is transformed with a nucleic acid encoding a suppressor of a post transcriptional gene silencing process.

94. A plant cell comprising a recombinant composition, wherein the recombinant composition comprises at least one recombinant amplicon comprising a nucleic acid encoding at least one signal peptide or at least one transit peptide, or both, operably linked to a nucleic acid encoding a polypeptide, wherein the polypeptide is expressed while a suppressor of a post transcriptional gene silencing process is active.

95. The plant cell of claim 94, wherein the transit peptide is a chloroplast-targeting transit peptide.

96. The plant cell of claim 94, wherein the recombinant composition further comprises at least one nucleic acid encoding a suppressor of a post transcriptional gene silencing process.

97. An isolated nucleic acid comprising a recombinant amplicon comprising a nucleic acid encoding at least one signal peptide or at least one transit peptide, or both, operably linked to a nucleic acid encoding a polypeptide, and optionally, further comprising a nucleic acid encoding a suppressor of a post transcriptional gene silencing process.
